(19) United States
(12) Patent Application Publication
(10) Pub. No.: US 2025/0327791 A1
(43) Pub. Date: Oct. 23, 2025

(54) METHODS OF TREATMENT BASED ON PATIENT SPECIFIC CLINICAL TRIAL MODELS AND ASSOCIATED MODELS AND USES

(71) Applicant: DUKE UNIVERSITY, Durham, NC (US)

(72) Inventors: Xiling SHEN, Durham, NC (US); Shiaowen David HSU, Durham, NC (US)

(21) Appl. No.: 18/609,720

(22) Filed: Mar. 19, 2024

Related U.S. Application Data
(63) Continuation of application No. 16/649,639, filed on Mar. 21, 2020, now abandoned.
(60) Provisional application No. 62/563,982, filed on Sep. 27, 2017.
(60) Provisional application No. 62/625,415, filed on Feb. 2, 2018.
(60) Provisional application No. 62/722,272, filed on Aug. 24, 2018.

(51) Int. Cl. G01N 33/50; C12N 5/09; G16H 10/20; G16H 50/50

(52) U.S. Cl. CPC G01N 33/50; G16H 10/20; G16H 50/50

(57) ABSTRACT

Methods of treatment and patient specific clinical trials are disclosed. According to an aspect, a method includes generating a patient specific tumor model. The method also includes testing one or more drugs on the patient specific tumor model. Further, the method includes treating a patient based on the results of the patient specific tumor model tests.

### CROSS REFERENCE TO RELATED APPLICATIONS

This U.S. Utility Patent Application claims priority to U.S. patent application Ser. No. 16/649,639, filed Mar. 21, 2020 and titled “PATIENT SPECIFIC CLINICAL TRIALS AND ASSOCIATED METHODS OF TREATMENT” which claims priority to PCT International Application No. PCT/US2018/053236, filed Sep. 27, 2018, and titled “PATIENT SPECIFIC CLINICAL TRIALS AND ASSOCIATED METHODS OF TREATMENT”, which claims priority to U.S. Patent Application No. 62/563,982, filed Sep. 27, 2017, and titled “Compositions, Methods and Systems for Model-Guided Individualized Clinical Trial (MICT) to Treat Drug Resistance after Standard-of-Care (DRASC)”; U.S. Patent Application No. 62/625,415, filed Feb. 2, 2018, and titled “INHIBITION OF FGFR AND MEK PATHWAYS TO TREAT COLORECTAL CANCER AND ITS LIVER METASTASIS”; and U.S. Patent Application No. 62/722,272, filed Aug. 24, 2018, and titled “DEVELOPMENT OF A RAPID ORGANOID THERAPEUTIC ASSAY (ROTA) TO GUIDE THERAPY IN PATIENTS WITH CANCER”, the content of which are incorporated herein by reference in their entireties.

### FEDERALLY SPONSORED RESEARCH OR DEVELOPMENT

This invention was made with government support under Federal Grant No. 5R35GM122465 awarded by National Institutes of Health (NIH). The government has certain rights to this invention.

### REFERENCE TO A “SEQUENCE LISTING,” APPENDIX SUBMITTED IN AN XML FILE FORMAT

This application contains a ST.26 sequence listing appendix. It has been submitted electronically via EFS-Web as an XML file entitled “210-100-UTIL2.xml”. The ST.26 sequence listing is 6,288 bytes in size and was created on 19 Mar. 2024. It is hereby incorporated by reference in its entirety.

### SEQUENCE DATA

I hereby state that the information recorded in computer readable form is identical to the written sequence listing below. I also hereby state that the computer readable copy and the electronic copy of the sequence listing submitted concurrently herewith contains no new matter, nor does it go beyond the disclosure of the application as filed.

### TECHNICAL FIELD

The presently disclosed subject matter relates generally to medical treatment. Particularly, the presently disclosed subject matter relates to patient specific clinical trials and associated methods of treatment.

### BACKGROUND

Despite a large investment of funds and efforts into cancer research, a cancer diagnosis is often terminal for the patient. It is believed that this largely stems from the fact that less than 1% of drugs developed in oncology proceed to the clinic.

Researchers look for drugs capable of eliminating a large variety of cancers across a large variety of patients. Cancer, however, is a personal disease that is different in every patient. Standard of care treatment for metastatic colorectal cancer, for example, consists of treatment with a combination of 5-FU and either oxaliplatin or irinotecan. However, more than half of patients do not respond to the first therapy chosen. This group of patients is usually treated with the unselected standard of care combination but this is only successful in at most 50% of patients. Although genomic based technologies such as next generation sequencing are currently being applied to look for actionable alterations, such as RAS mutation and the use if anti-EGFR (epidermal growth factor receptor), the fact is that the majority of identified cancer mutations are not targetable by drugs. However, there may be many potentially effective treatments for an individual patient, such as repurposing drugs that have already been FDA approved for another cancer type or drugs being tested in ongoing clinical trials, or compounds still yet to be clinically evaluated such as the ones listed in the National Cancer Institute (NCI) Cancer Therapy Evaluation Program (CTEP). Currently, these potentially lifesaving drugs languish for lack of clinical trial funding from drug companies unwilling to spend hundreds of millions of dollars on drugs that may not be widely successful with treating a variety of cancers in a large number of patients. Accordingly, there is a need for a less expensive and efficient clinical trial process.

Precision medicine, pairing the right therapy with the right patient at the right time, has been suggested as a technique of improved efficacy with minimal toxicity. However, the clinical applicability of patient derived preclinical cancer models (PDMCs) such as organoids, cell lines or patient derived xenografts (PDXs) is limited due to their months long development time. Accordingly, there is a need for improved preclinical models capable of improving both drug development and precision medicine.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is a flow diagram of an example clinical trial in accordance with embodiments of the present disclosure;

FIG. 2 is a flow diagram of one embodiment of the disclosure;

FIG. 3A is a flow diagram of one embodiment of the disclosure;

FIG. 3B is an image displaying histological features of PDX and matched cell lines in one embodiment of the disclosure;

FIG. 4A are tables listing the results of high-throughput drug screens in one embodiment of the disclosure;

FIG. 4B are graphs displaying matched PDX tumor data in accordance with embodiments of the present disclosure;

FIG. 5 are tables listing the results of mined high-throughput drug screen data in one embodiment of the disclosure;

FIG. 6A is a Venn diagram showing the overlap in pathways targeted by various cancer drugs;

FIG. 6B are graphs showing results of cell line drug screens in embodiments of the present disclosure;

FIG. 7A are graphs displaying the ponatinib IC₅₀ for various cell lines in one embodiment of the disclosure;

FIG. 7B is an image of a gel displaying FGFR expression western blot data for various cell lines in accordance with embodiment of the present disclosure;

FIG. 7C is an image displaying the major signaling pathways downstream of FGFR;

FIG. 8 is an image of a gel displaying expression data of various proteins from various cell lines pre and post ponatinib treatment measured through western blot in one embodiment of the disclosure;

FIG. 9A is a graph displaying the results of ponatinib treatment of a PDX model in accordance with embodiments of the present disclosure;

FIG. 9B is a graph displaying the results of ponatinib treatment of a PDX model in accordance with embodiments of the present disclosure;

FIG. 9C is a graph displaying the results of ponatinib treatment of a PDX model in accordance with embodiments of the present disclosure;

FIG. 10 is a flow diagram of an example treatment plan in accordance with embodiments of the present disclosure;

FIG. 11 is an image displaying the histological features of an example patient tumor, matching organoids and PDX in one embodiment of the disclosure;

FIG. 12 is a flow diagram in accordance with embodiments of the present disclosure;

FIG. 13 is a flow diagram in accordance with embodiments of the present disclosure;

FIG. 14A is a graph displaying sensitivity data of various organoids to various concentrations of oxaliplatin in accordance with embodiments of the present disclosure;

FIG. 14B is a graph displaying sensitivity data of various organoids to oxaliplatin in accordance with embodiments of the present disclosure;

FIG. 14C is a graph displaying sensitivity data of various organoids to oxaliplatin in accordance with embodiments of the present disclosure;

FIG. 14D is a graph displaying sensitivity data of various organoids to oxaliplatin in accordance with embodiments of the present disclosure;

FIG. 14E is a graph displaying sensitivity data of various organoids to oxaliplatin in accordance with embodiments of the present disclosure;

FIG. 14F graph displaying sensitivity data of various organoids to oxaliplatin in accordance with embodiments of the present disclosure;

FIG. 15 is a graph displaying sensitivity data of various organoids to 1 μM oxaliplatin;

FIG. 16A is a graph displaying sensitivity data of oxaliplatin resistant organoids;

FIG. 16B is a graph displaying sensitivity data of oxaliplatin resistant PDX models;

FIG. 16C is a graph displaying sensitivity data of oxaliplatin resistant organoids;

FIG. 16D is a graph displaying sensitivity data of oxaliplatin resistant PDX models;

FIG. 16E is a graph displaying sensitivity data of oxaliplatin resistant organoids;

FIG. 16F is a graph displaying sensitivity data of oxaliplatin resistant PDX models;

FIG. 17A is a graph displaying sensitivity data of oxaliplatin susceptible organoids;

FIG. 17B is a graph displaying sensitivity data of oxaliplatin resistant PDX models;

FIG. 17C is a graph displaying sensitivity data of oxaliplatin resistant organoids;

FIG. 17D is a graph displaying sensitivity data of oxaliplatin resistant PDX models;

FIG. 18A is a graph displaying irinotecan sensitivity data of organoids;

FIG. 18B is a graph displaying irinotecan sensitivity data of PDX models;

FIG. 18C is a graph displaying irinotecan sensitivity data of organoids;

FIG. 18D is a graph displaying irinotecan sensitivity data of PDX models;

FIG. 19 is a table showing various optimized growth factor combinations

FIG. 20A is a picture showing histological data for three different organoids;

FIG. 20B is graphs showing the oxaliplatin IC50 for three different organoids;

FIG. 21A are graphs showing 5-FU and SN38 cell viability data;

FIG. 21B are graphs showing 5-FU and SN38 IC50 data;

FIG. 21C are graphs showing 5-FU and SN38 IC50 data;

FIG. 22A are graphs showing the results of ATAC Seq data;

FIG. 22B are graphs showing the results of RT-PCR;

FIG. 23 are graphs showing organoid and PDX cell viability data; and

FIG. 24 is a graph showing organoid cell viability data.

### SUMMARY

Disclosed herein are patient specific clinical trials and associated methods of treatments. According to an aspect, a method includes generating a patient specific tumor model. The method also includes testing one or more drugs on the patient specific tumor model. Further, the method includes treating a patient based on the results of the patient specific tumor model tests.

According to an aspect, patient specific information is entered into a computational model. According to an aspect a patient is treated based on the results of the patient specific tumor model tests and the computational model. According to an aspect a cancer patient is treated with an effective amount of an FGFR inhibitor. According to an aspect a cancer patient is treated with an effective amount of a substance that targets the MEK/RAS/RAF/ERK pathway. According to an aspect a cancer patient is treated with an effective amount of a substance that targets the PI3K/AKT/mTOR pathway. According to an aspect a cancer patient is treated with an effective amount of a substance that targets the PI3K/AKT/mTOR and the MEK/RAS/RAF/ERK pathways. According to an aspect a cancer patient is treated with an effective amount of an FGFR inhibitor and a substance that targets the MEK/RAS/RAF/ERK and the PI3K/AKT/mTOR pathways. According to an aspect a patient's tumor is searched for FGFR mutations and if mutations are present the patient is treated with a substance that targets the MEK/RAS/RAF/ERK pathway. According to an aspect a patient's tumor is searched for FGFR mutations and if mutations are found the patient is treated with a substance that targets the PI3K/AKT/mTOR pathway. According to an aspect a patient's tumor is searched for FGFR mutations and if mutations are found the patient is treated with an FGFR inhibitor. According to an aspect an organoid comprising tumor immune, endothelial and mesenchymal cells is disclosed. According to an aspect, a patient derived tumor organoid is created by obtaining a biopsy of a patient's cancer, digesting the biopsied cells, and seeding the cells such that tumor immune, endothelial and mesenchymal cells are included in the organoid.

### DETAILED DESCRIPTION

The following detailed description is made with reference to the figures. Exemplary embodiments are described to illustrate the disclosure, not to limit its scope, which is defined by the claims. Those of ordinary skill in the art will recognize a number of equivalent variations in the description that follows.

As referred to herein, a patient specific clinical trial system refers to a system that allows for the testing of drugs or other treatment methods on a disease model closely matching that of the patient. Non-limiting examples include a cell line derived from a patient's tumor, a PDX derived from a patient's tumor, a PDX derived from a cell line that was derived from a patient's tumor, organoid culture derived from a patient's tumor, or a cell line that was derived from a PDX that was derived from a patient's tumor.

As referred to herein, a PDX is a patient derived xenograft. As a non-limiting example, a tumor grown from biopsy derived cancer cells injected subcutaneously into a mouse flank.

As referred to herein, an organoid is a cell model designed to more closely resemble the original cellular environment when compared to normal 2D cell culture. In a non-limiting example a tumor model grown from tumor stem cells that closely mimics the original tumors cellular environment may be an organoid.

As referred to herein, genome editing is the process of replacing or removing part or all of a genome. CRISPER in a non-limiting example would be a genome editing procedure.

Unless otherwise noted all experiments were carried out using the following materials and methods which are here presented as examples and not limiting embodiments. All equivalent variants are contemplated as part of the presently disclosed subject matter. An Echo Acoustic Dispenser provided automated liquid handling for drug addition while cell plating was performed by a Thermo Fisher Well Mate and assays used a Clarioscan plate reader. The drugs assayed were stamped to the cell plates immediately prior to cell plating at a final concentration of 1 μM. The drug pre-coated plates were plated with 500-1000 cells/well. 72 hours after cell plating cell viabilities were assessed via a CellTiter-Glo Luminescent Cell Viability Assay.

For in vitro screening, cell lines were cultured in DMEM+10% FBS+1% Penicillin/Streptomycin and plated in drug free medium. Ponatinib solubilized in DMSO was added to cell lines containing between 3000-6000 cells that had been incubated at 37° C. for 24 hours. Each cell line was exposed to seven different drug combinations between 1.6 nM and 25 μM. Five replicates were used for each drug concentration. 72 hours after drug addition cell viability assay and IC50 values were calculated for each cell line using GraphPad Prism software.

150 μL of 150 mg/ml homogenized PDX tissue-PBS suspension was subcutaneously injected into the right flanks of 5 female and 5 male ten week old mice. The experimental group received an oral dosing of 30 mg/kg ponatinib once tumor volumes reached approximately 150 mm³. Tumor volume measurement were performed every other day using calipers and tumor size was calculated using the formula (length x(width)²/2. 2-way ANOVA analysis was used to compare the tumor size between control groups and treatment groups. A p value<0.05 was considered statistically significant.

Western blot analysis was performed by lysing a total of 100,000 cells in protease and phosphatase inhibitor cocktail supplemented radioimmnoprecipitation assay lysis buffer. 50 μg of RIPA lysate was electrophoretically separated at 200V on 4-20% sodium dodecyl sulfate polyacrylamide gels. Membranes were blocked in StartingBlock T20 for one hour at room temperature, incubated in primary antibody diluted in StartingBlock T20 overnight at 4° C. with rocking and transferred onto nitrocellulose membranes at 50V for two hours. Membranes were washed for five minutes three times each in PBS+0.05% Tween-20 and incubated in corresponding Horse Radish Peroxidase conjugated secondary antibodies. All antibodies were used at 1:1000 dilutions.

RNA-seq libraries were prepared and sequenced in Illumina HiSeq 4000 with 150 bp paired-end reads aligned to human genome hg19. 150 bp PE reads were first aligned using the STAR-2pass method with default parameters. The output SAM files were processed using Picard to add read group, sort, mark duplicates and index. Identified variants were annotated using SnpEff and GTAK was used for variant calling.

In embodiments, organoids are prepared by mincing a 0.2-0.3mm³ tissue sample into <2 mm³ pieces. Samples are then digested in 5 mL of DMEMF-12+Penicillin Streptomycin+Rock inhibitor Y-27632 along with 20 μL of 0.25% Trypsin/EDTA for an hour with manual inversion every 10 minutes. After being spun down at 1500 RPM the pellet is washed with 5 mL of 10% FBS. During each of 3 washes the material is pipetted slowly about fifteen times. After each wash supernatant is collected and passed through a 70 μm cell strainer. Collected washes are spun down for five minutes at 1500 RPM and the pellet mixed with a 4:1 mixture of Matrigel®/PBS and plated. After the Matrigel® solidifies 1 mL of media is added to each well.

In embodiments for rapid treatment guiding screening, Organoids incubated for about 3-4 days at 37° C. have media removed and 1 ml of PBS added to each well to detach the matrigel. Collected matrigel is spun for 7 minutes at 1500 RPM. The pellet is collected and resuspended in 300 μL of PBS. 50 μL of this mixture is then mixed with 50 μL of a 1:1 mixture of matrigel/PBS. 5 μL of this mixture is then added to the center of each well in a 96 well plate and the plate incubated until the matrigel solidifies. Typically, this does not take longer than 10-15 minutes. After 90 μL of media is added and the plate is incubated at 37° C. for 24 hours, 5 μL of the tested drug is added to each well. Example concentrations, such as 100 nM, 1 μM and 10 μM concentrations, may be tested in triplicate. After the plate is incubated at 37° C. for 48 hours, 40 μL of Cell Titer Glo for organoids is added to each well to determine drug sensitivity.

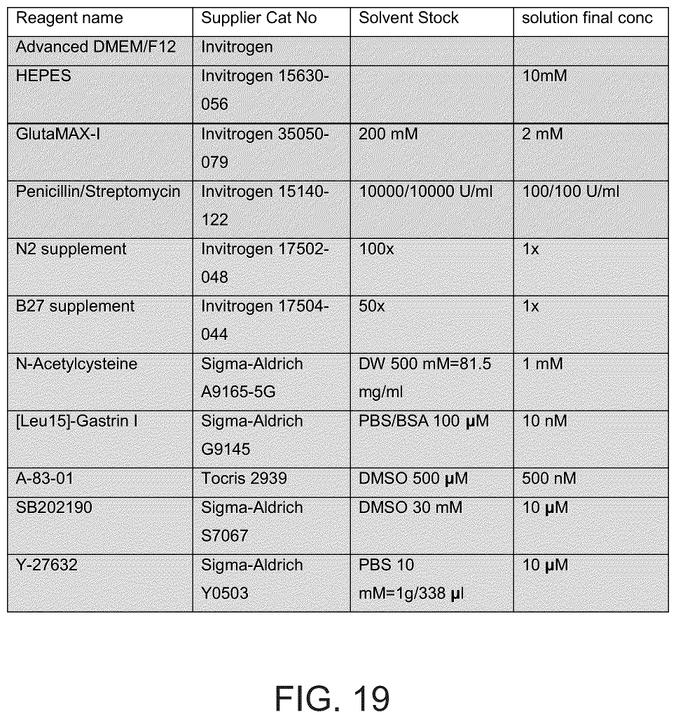

In embodiments organoids were created by embedding single cells in Matrigel on ice and seeding the cells in 48 well plates. After the Matrigel was polymerized for 10 minutes at 37° C. basal culture medium was overlaid containing at least one of the optimized growth factor combinations in FIG. 19.

Genome editing studies may be conducted by generating single-guide RNA libraries for targeted genomic sites. The libraries may be cloned into lentiviral expression vectors for delivery. Intestinal organoid cells may be transduced at a low MOI of 0.8 so that delivery of one sgRNA per cell is assured. After a 12-15 day selection period two target populations of Lgr5-GFP plus dsRed double positive cells (ISCs) and dsRed only positive cells (non-ISCs) may be purified and collected using FACS and then subjected to deep sequencing so that the relative abundance of each sgRNA in both populations may be identified. Significant pathways and underlying mechanisms may be identified through sgRNA annotation and gene ontology enrichment analysis.

In an embodiment predesigned sequence specific shRNA vectors, pLKO 1-puro vectors, and lentiviral packaging vectors in the form of bacterial glycerol stock were used. Plasmids were extracted as known in the art and cells were transfected with the plasmids to package lentiviruses using commercial transfection reagents as known in the art. The collected lentiviruses were used to silence or mock silence genes of interest. Puromycin was added to the cell culture medium for selection.

Real-time-Reverse-Transcription was carried out by extracting RNA using Qiagen's RNeasy Kit. cDNA was synthesized using QuantiTect Reverse Transcription Kit. PCR reactions were prepared using QuantiFast SYBR Green PCR Kit. Real time-RT-PCR was performed with a two step cycling protocol, with a denaturation step at 95° C. and a combined annealing/extension step at 60° C.

PDX studies accompanying the organoid studies were developed as described previously and in Uronis J M, Osada T, McCall S, Yang X Y, Mantyh C, Morse M A, et al. Histological and molecular evaluation of patient-derived colorectal cancer explants. PloS one 2012; 7:e38422, and Kim M K, Osada T, Barry W T, Yang X Y, Freedman J A, Tsamis K A, et al. Characterization of an oxaliplatin sensitivity predictor in a preclinical murine model of colorectal cancer. Molecular cancer therapeutics 2012; 11:1500-1509. Both of these references are hereby incorporated in their entirety. 6-8 week old NOD/SCID-beige mice were used and the tumors were measured twice a week as described above. Once tumors reached a size of 250 mm³ mice were treated with either 10 mg/kg oxaliplatin or 20 mg/kg irinotecan weekly via IP (intraperitoneal injection) for three weeks with saline used as a control. PDX tumor sizes were recorded and one-way ANOVA analysis were carried out as described in the references above to determine TGI (tumor growth inhibition.

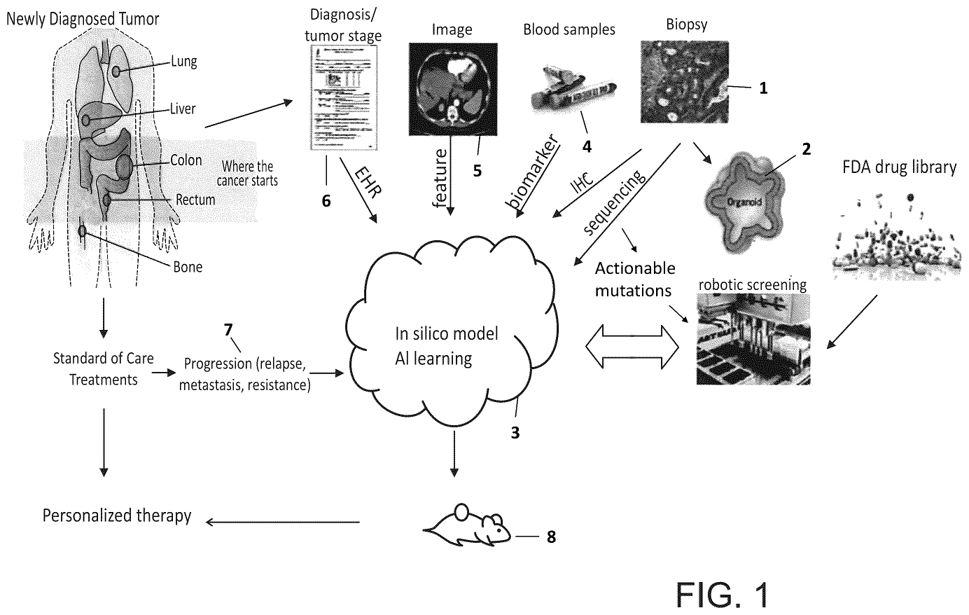

In accordance with embodiments of the present disclosure, compositions methods and systems for model-guided individualized clinical trials (MICT) are disclosed. FIG. 1 illustrates a flow diagram of an example clinical trial in accordance with embodiments of the present disclosure. Referring to FIG. 1, a biopsy is taken of the patients cancer and specific drugs tested against ex vivo and/or in vivo models derived from the patient's tumor. In embodiments, computational models (in silico, Baysian) may be used to pre-screen the drug library and/or predict therapeutic efficacy. In embodiments, machine learning techniques may be used to either train the model on standard data before use or improve the model over multiple clinical trials. In embodiments, a biopsy 1 is taken of the patient's tumor and a cell line is grown from the patient's tumor biopsy. In embodiments, an organoid 2 is grown from the patient's tumor biopsy. In embodiments, an organoid 2 and a cell line are grown from the patient's tumor biopsy 1. In embodiments, the drugs contained in the NCI CTEP database are tested on the cell line derived from the patient's tumor biopsy. In embodiments, the drugs contained in the NCI CTEP database are tested on the organoid derived from the patient's tumor biopsy. Although an NCI CTEP database is described by example, it should be understood that any database of drugs may be used.

In embodiments, a computational model 3 assists in the clinical trial. In embodiments, biopsy IHC or biopsy sequencing data are entered into the computational model. In embodiments, biomarkers from patient blood samples 4 are entered into the computational model. In embodiments, features derived from patient imaging data 5 are entered into the computational model. In embodiments, diagnostic information 6 is entered into the computational model. In embodiments, patient disease progression information 7 may be entered into the computational model. In embodiments, one, multiple or all information from the following group: biopsy IHC, biopsy sequencing data, biomarkers 4, features derived from patient imaging data 5, diagnostic information 6, patient disease progression information 7, medical images, histology and/or immunohistochemistry images from tumor biopsies, and genetic mutations present in the tumor are entered into the computational model. In embodiments, the computational model helps screen and select the best individual or combinatorial drug regimens. In embodiments, patient tumors with stroma may be directly implanted into the flanks of immunodeficient mice 8. In embodiments, new patient information, new drug libraries, and new patient-derived models are continuously incorporated.

In embodiments, drug candidates may be tested in patient-derived tumor animal models. In embodiments, drug candidates may be tested in an orthotopic-metastasis transplant model. In embodiments, drug candidates may be tested in a blastocyst-injection chemokine-targeting model. In embodiments, drug candidates are tested in one, multiple, or all of the following animal models: orthotopic metastasis, blastocyst injection, chemokine-targeting.

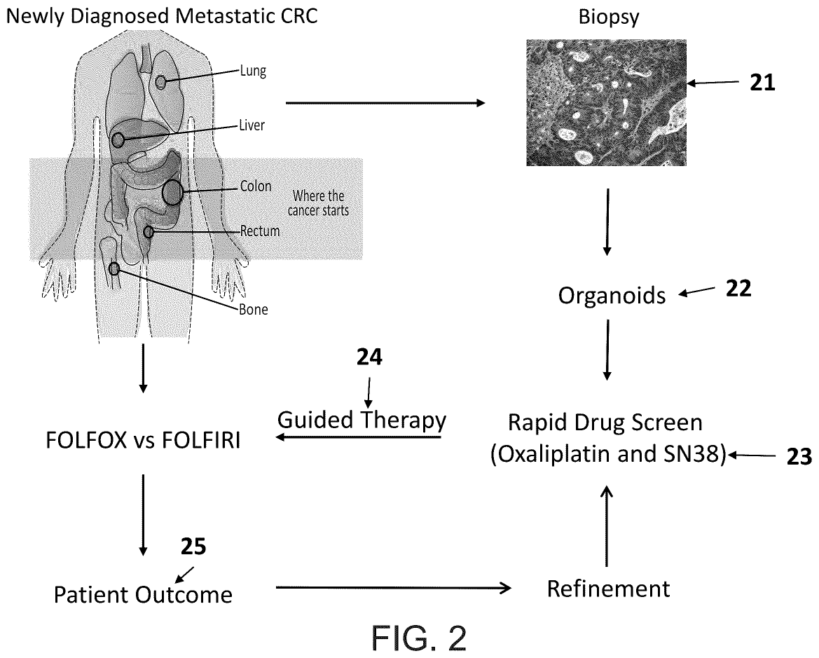

In an example, as shown in FIG. 2, metastatic CRC may be biopsied 21, organoids created 22, and rapid drug screens 23 may guide therapy 24. Patient outcomes may be used to refine 25 the rapid drug screen as well.

In embodiments ten patients with CRC liver metastasis undergo biopsy of their liver lesion and CRC liver metastasis diagnosis verification through pathology. The patients' chest, abdomen and pelvis are then CT scanned for measurement of tumor size and staging. Patient specific organoids are then generated and an assay performed to determine oxaliplatin sensitivity. While this is being carried out patients are treated with FOLFOX for 2 months with restaging performed using CT scans of the chest, abdomen, and pelvis at the end of neoadjuvant chemotherapy. Patient derived xenografts, will be produced and genomic analysis and drug screens carried out using remaining patient biopsy sample.

In embodiments patients whose organoids are sensitive to oxaliplatin will be assigned to FOLFOX while patients' whose organoids are resistant to oxaliplatin will be assigned to either FOLFOX or FOLFIRI. In embodiments all patients involved in the study will have life expectancies greater than 12 weeks. In embodiments all enrolled patients will have no previous treatment. In embodiments all patients will have an ECOG performance status of 0 to 2. In embodiments the results of the organoid oxaliplatin assay will be correlated with patient response to FOLFOX. In embodiments staging and restaging at end of neoadjuvant chemotherapy will be performed by MRI.

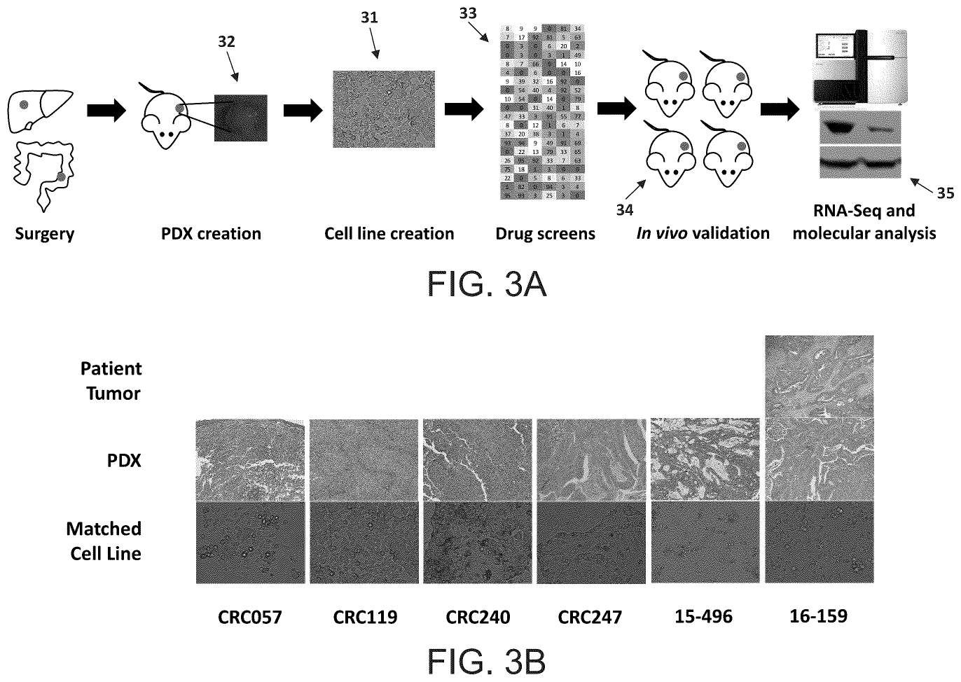

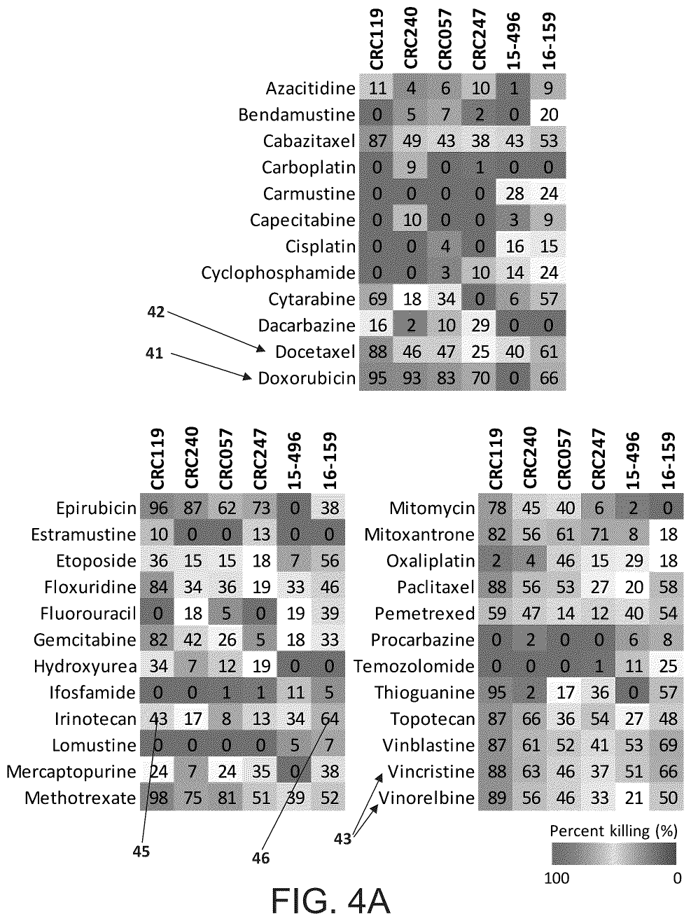

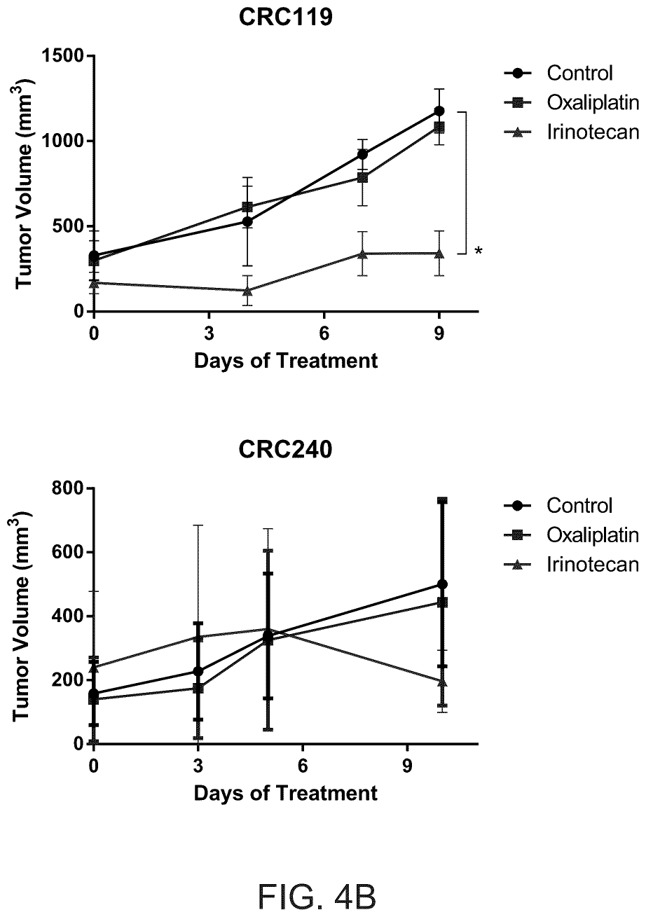

In embodiments, a PDMC can be developed for patients undergoing cancer treatment as shown in FIG. 3A. In this embodiment matching cells lines 31 and PDXs are created 32. These can be developed as described in the Uronis and Kim papers previously incorporated by reference. Drugs may subsequently be screened using these cell lines 33 the results validated in vivo 34 and RNA-Seq and molecular analysis 35 used. As a non-limiting example, CRC057, CRC119, CRC240, CRC247 15-496, and 16-159 were derived from patient colorectal cancers. It should be understood by those of skill in the art that any suitable type of cancer sample may have been taken. Histological features of the PDXs and matched cell lines are shown in FIG. 3B. High-throughput drug screens, including 119 FDA-approved drug compounds, were performed using the patient-derived cell lines. Any suitable type of high or low throughput drug screen of any FDA approved or non-FDA approved drug may be performed on the cell lines. As shown in FIG. 4A, the CRC cell lines were sensitive to anthracyclines 41, taxanes 42, and vinca alkaloids 43. 88%, 95%, 88, and 89% of CRC119 were killed by docetaxel 42, doxorubicin 41, and the vinca alkaloids vincristine and vinorelbine 43 respectively. 46%, 93%, 63% and 56% of CRC240 were killed by docetaxel 42, doxorubicin 41, and the vinca alkaloids vincristine and vinorelbine 43 respectively. 47% 83%, 46% and 46% of CRC057 were killed by docetaxel 42, doxorubicin 41, and the vinca alkaloids vincristine and vinorelbine 43 respectively. 25%, 70%, 37%, and 33% of CRC247 were killed by docetaxel 42, doxorubicin 41, and the vinca alkaloids vincristine and vinorelbine 43 respectively. Only CRC057 was found to be sensitive to the standard of care cytotoxic chemotherapeutic agent oxaliplatin 44 with 46% of the cells being killed. CRC119 45 and 16-159 46 were sensitive to the standard of care cytotoxic chemotherapeutic agent irinotecan with 43% and 64% of cells killed respectively. Matched PDX tumors were used for in vivo validation as shown in FIG. 4B.

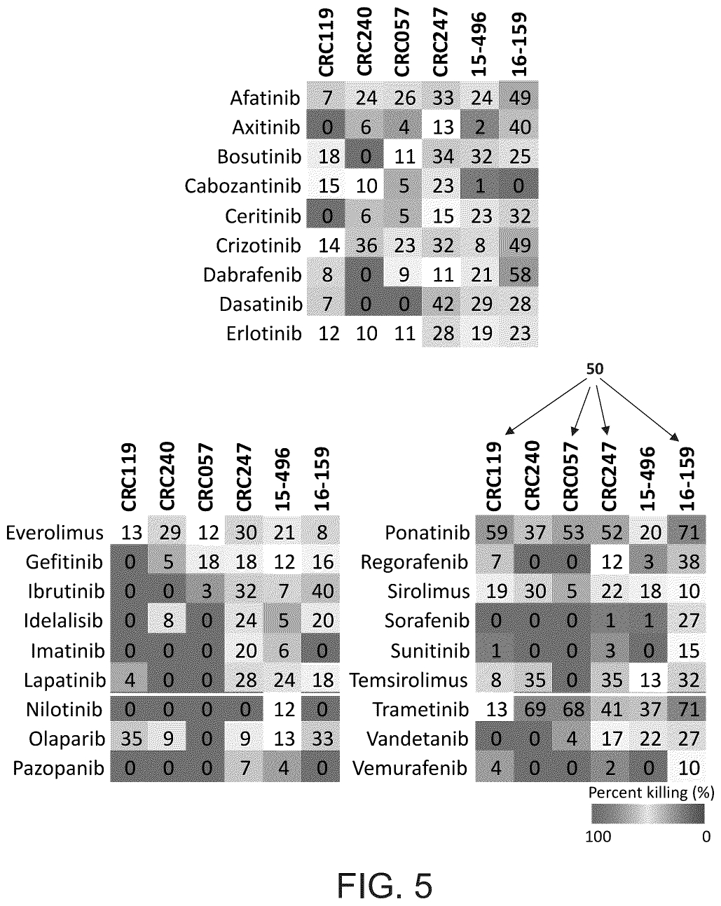

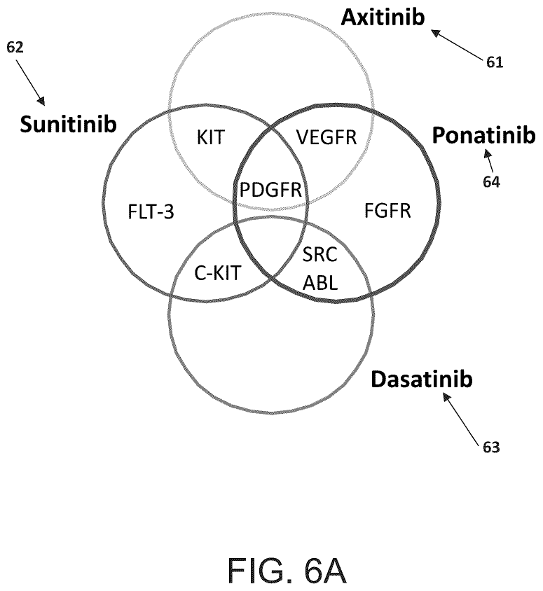

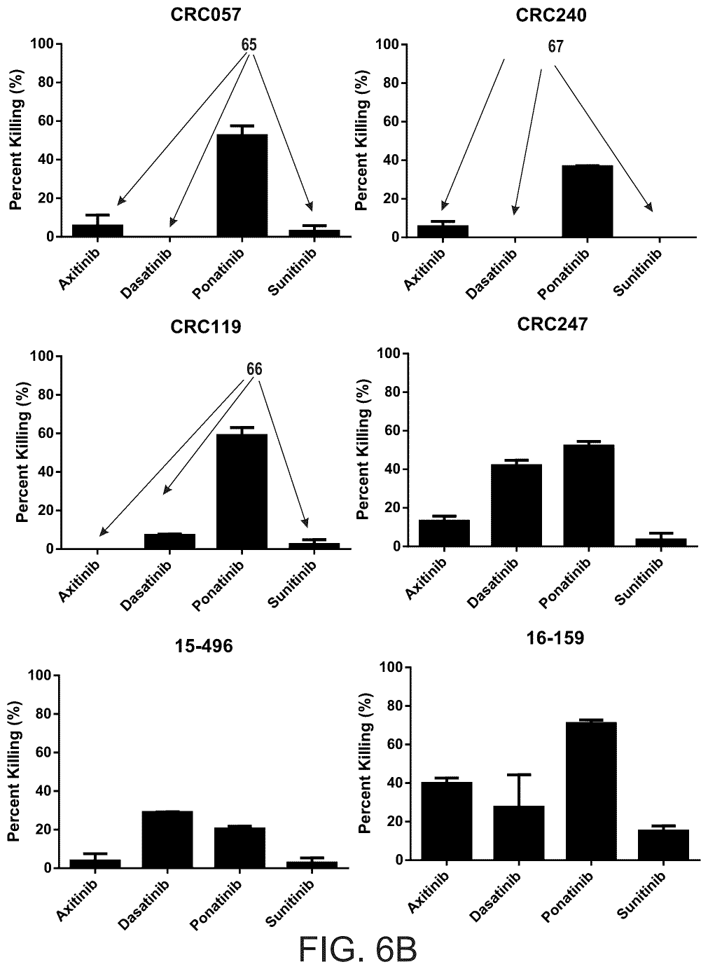

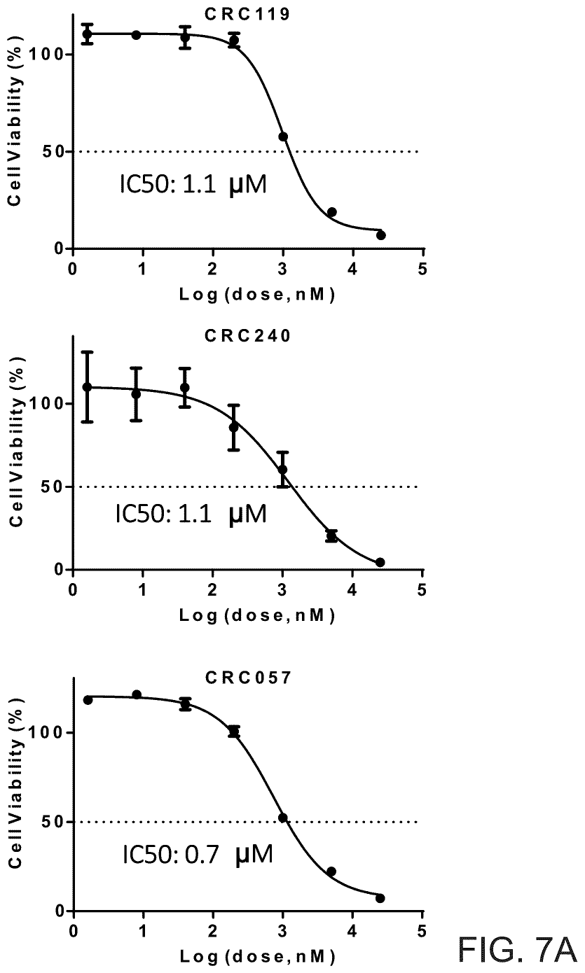

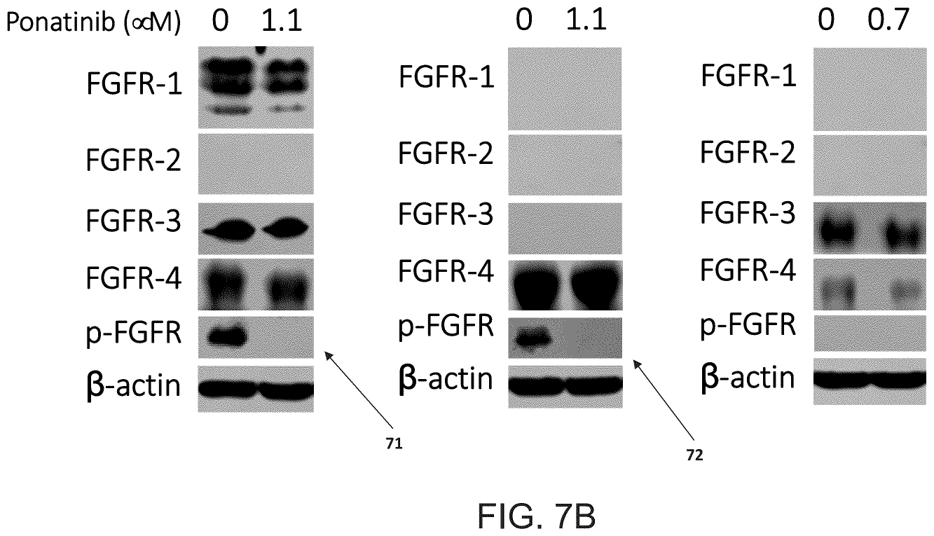

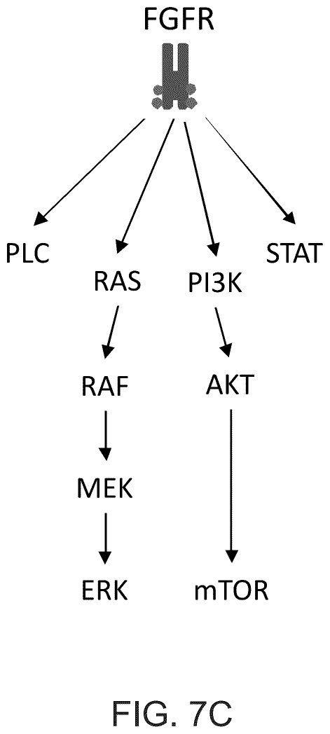

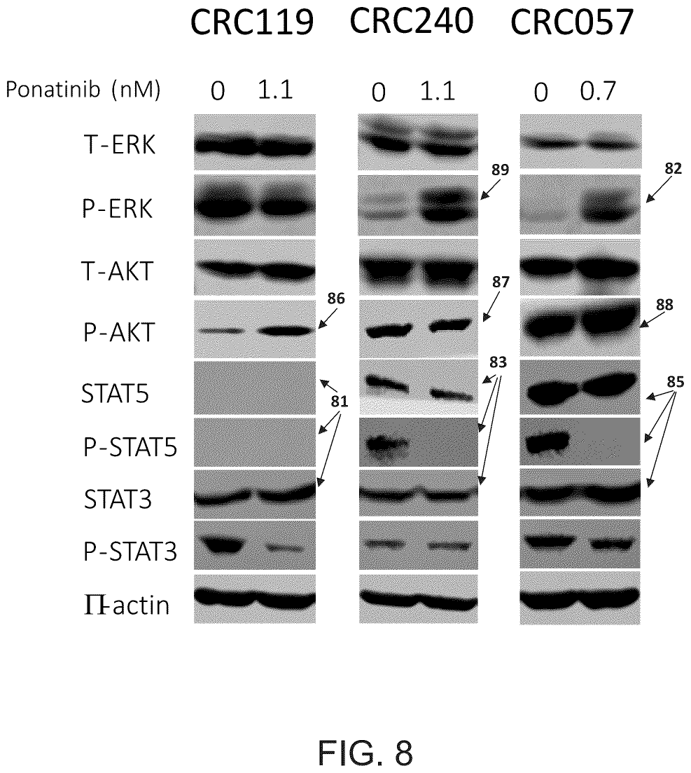

As shown in FIG. 5, mined drug screen data shows that only ponatinib inhibits growth by ≥50% in 4/6 cell lines 50. Reanalyzing the screen data, FIG. 6A identified axitinib 61, sunitinib 62, and dasatinib 63 as targeting similar pathways as ponatinib 64. Unexpectedly, as shown in FIG. 6B, axitinib, sunitinib and dasatinib were resisted by CRC057 65, CRC 119 66, and CRC 240 67 suggesting that ponatinib targets FGFR in these cell lines. As shown in FIG. 7A the ponatinib IC₅₀ was found to be 0.7 82 M for CRC057, 1.1 μM for CRC 119 and 1.1 μM for CRC240. Western blot analysis with FGFR antibodies pre and post ponatinib treatment, FIG. 7B, demonstrates that phosphorylated FGFR was inhibited in CRC119 71 and CRC240 72. Pre and post ponatinib treatment the major signaling pathways downstream of FGFR, FIG. 7C, not only show a decrease in STAT expression FIG. 8 in CRC119 81, CRC240 83, and CRC057 85 but an increase in p-AKT expression in CRC 119 86, CRC240 87, and CRC057 88. Expression of p-ERK increased in CRC240 89, and CRC057 82 as well.

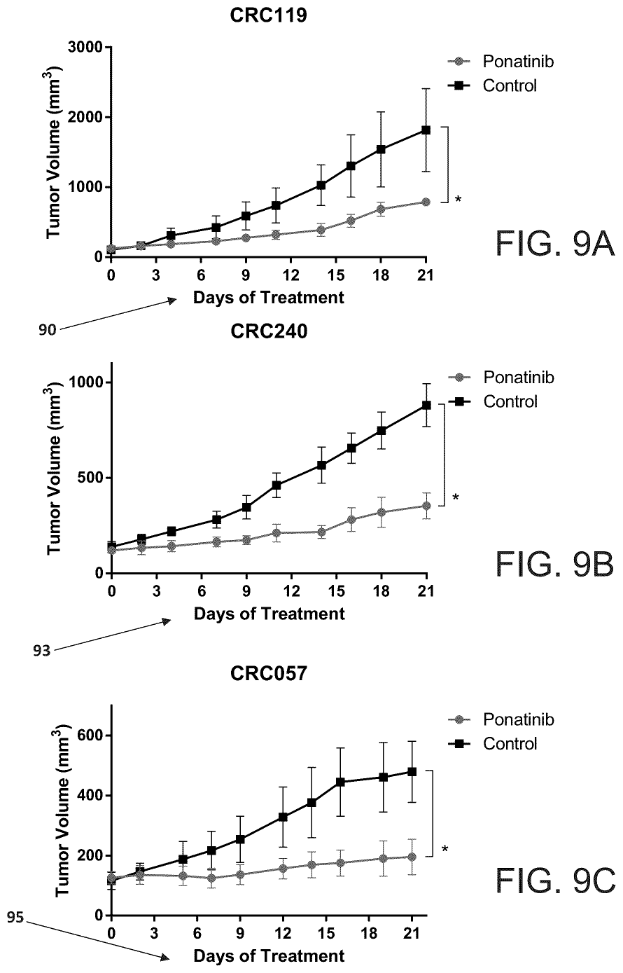

These results were validated in vivo by injecting matched PDX models of CRC119, CRC 240, and CRC057 into the flanks of mice as described in the Uronis and Kim papers previously incorporated and treating the mice with 30 mg/kg of oral ponatinib five times a week. As shown in FIG. 9, CRC119 90, CRC240 93 and CRC057 95 were all sensitive to ponatinib.

In embodiments, the MEK/RAS/RAF/ERK pathway is targeted for colorectal cancer treatment. In embodiments, the MEK/RAS/RAF/ERK pathway is targeted for treatment of colorectal cancer with liver metastasis. In embodiments, the MEK/RAS/RAF/ERK pathway is targeted by an inhibitor. In embodiments, the MEK/RAS/RAF/ERK pathway is targeted by an activator. In embodiments, the PI3K/AKT/mTOR pathway is targeted for colorectal cancer treatment. In embodiments, the PI3K/AKT/mTOR pathway is targeted for colorectal cancer treatment with liver metastasis. In embodiments, the PI3K/AKT/mTOR pathway is targeted by an inhibitor. In embodiments, the PI3K/AKT/mTOR pathway is targeted by an activator. In embodiments, both the MEK/RAS/RAF/ERK and the PI3K/AKT/mTOR pathways are targeted by an inhibitor. In embodiments, both the MEK/RAS/RAF/ERK and the PI3K/AKT/mTOR pathways are targeted by an activator. In embodiments, the MEK/RAS/RAF/ERK pathway is targeted by an activator and the PI3K/AKT/mTOR pathway is targeted by an inhibitor. In embodiments, the MEK/RAS/RAF/ERK pathway is targeted by an inhibitor and the PI3K/AKT/mTOR pathway is targeted by an activator. In embodiments, the MEK/RAS/RAF/ERK and the PI3K/AKT/mTOR pathways are targeted for colorectal cancer. In embodiments, the MEK/RAS/RAF/ERK and the PI3K/AKT/mTOR pathways are targeted for colorectal cancer with liver metastasis. In embodiments, FGFR is inhibited and the MEK/RAS/RAF/ERK pathway is targeted for cancer treatment. In embodiments, FGFR is inhibited and the MEK/RAS/RAF/ERK pathway is targeted for colorectal cancer treatment. In embodiments, FGFR is inhibited and the MEK/RAS/RAF/ERK pathway is targeted for colorectal cancer with liver metastasis. In embodiments, FGFR is inhibited and the PI3K/AKT/mTOR pathway is targeted for cancer treatment. In embodiments, FGFR is inhibited and the PI3K/AKT/mTOR pathway is targeted for colorectal cancer treatment. In embodiments, FGFR is inhibited and the PI3K/AKT/mTOR pathway is targeted for colorectal cancer treatment with liver metastasis. In embodiments, FGFR is inhibited and the PI3K/AKT/mTOR and MEK/RAS/RAF/ERK pathways are targeted for colorectal cancer treatment. In embodiments, FGFR is inhibited and the PI3K/AKT/mTOR and MEK/RAS/RAF/ERK pathways are targeted for colorectal cancer with liver metastasis.

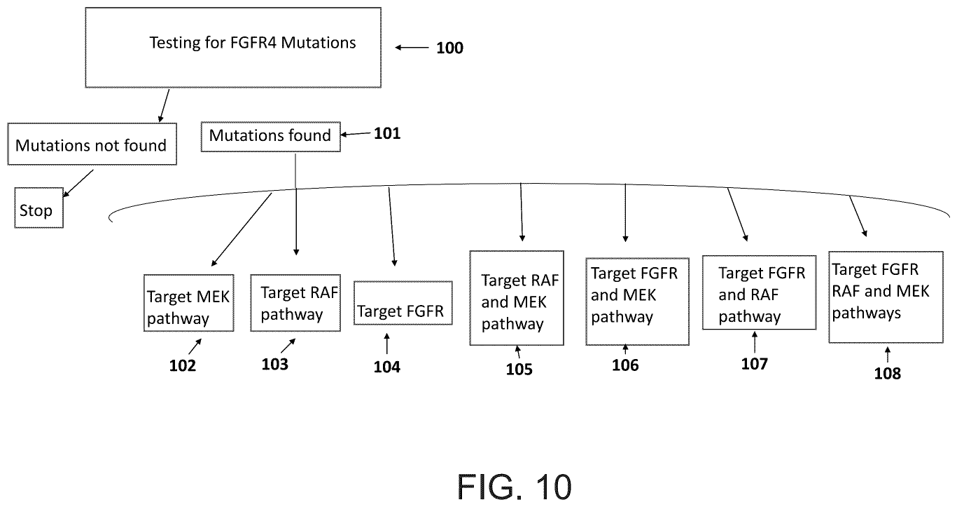

RNA-Seq data found the P136L mutation in FGFR4 in all six patient derived cell lines. This mutation could be found using either SEQ ID. NO 1, SEQ ID NO 3 or SEQ ID NO 5 as forward primers, and either SEQ ID. NO 2, SEQ ID NO 4 or SEQ ID NO 6 as reverse primers. As would be obvious to one of ordinary skill in the art primers other than these could of course be used. Three of the cell lines contained the G388R mutation in FGFR4. In embodiments, shown in FIG. 10, FGFR mutations are searched for in a cancer patient 100. In embodiments, FGFR mutations are searched for using DNA sequencing. In embodiments, FGFR mutations are searched for using RNA sequencing. In embodiments, proteins are sequenced to look for FGFR mutations. In embodiments, FGFR mutations are searched for using PCR. In embodiments, FGFR mutations are searched for using micro arrays. In embodiments, FGFR mutations are searched for using next generation sequencing. In embodiments, the P136L mutation is searched for in FGFR4. In embodiments, the G388R mutation is searched for in FGFR4. In embodiments, FGFR mutations are searched for 100 and if found 101 the MEK/RAS/RAF/ERK pathway is targeted 102 for colorectal cancer treatment. In embodiments, FGFR mutations are searched for 100 and if found 101 the MEK/RAS/ERK pathway is targeted for colorectal cancer treatment with liver metastasis. In embodiments, FGFR mutations are searched for 100 and if found 101 the PI3K/AKT/mTOR pathway 103 is targeted for treatment of colorectal cancer. In embodiments, FGFR mutations are searched for and if found the PI3K/AKT/mTOR is targeted for treatment of colorectal cancer with liver metastasis. In embodiments, FGFR mutations are searched for and if found FGFR is inhibited 104 as a treatment for colorectal cancer. In embodiments, FGFR mutations are searched for and if found FGFR is inhibited as a treatment for colorectal cancer with liver metastasis. In embodiments, FGFR mutations are searched for and if found the PI3K/AKT/mTOR and MEK/RAS/RAF/ERK pathways are targeted 105 for cancer treatment. In embodiments, FGFR mutations are searched for and if found the PI3K/AKT/mTOR and MEK/RAS/RAF/ERK pathways are targeted for colorectal cancer treatment. In embodiments, FGFR mutations are searched for and if found the PI3K/AKT/mTOR and MEK/RAS/RAF/ERK pathways are targeted for colorectal cancer with liver metastasis treatment. In embodiments, FGFR mutations are searched for and if found FGFR is inhibited and the MEK/RAS/ERK pathway is targeted 106 for cancer treatment. In embodiments, FGFR mutations are searched for and if found FGFR is inhibited and the MEK/RAS/RAF/ERK pathway is targeted for treatment of colorectal cancer. In embodiments, FGFR mutations are searched for and if found FGFR is inhibited and the MEK/RAS/RAF/ERK pathway is targeted for treatment of colorectal cancer with liver metastasis. In embodiments, FGFR mutations are searched for and if found FGFR is inhibited and the PI3K/AKT/mTOR pathway is targeted 107 for cancer treatment. In embodiments, FGFR mutations are searched for and if found FGFR is inhibited and the PI3K/AKT/mTOR pathway is targeted for treatment of colorectal cancer. In embodiments, FGFR mutations are searched for and if found FGFR is inhibited and the PI3K/AKT/mTOR pathway is targeted for treatment of colorectal cancer with liver metastasis. In embodiments, FGFR mutations are searched for and if found FGFR is inhibited and the PI3K/AKT/mTOR and MEK/RAS/RAF/ERK pathways are targeted 108 for cancer treatment. In embodiments, FGFR mutations are searched for and if found FGFR is inhibited and the PI3K/AKT/mTOR and MEK/RAS/RAF/ERK pathways are targeted for colorectal cancer treatment. In embodiments, FGFR mutations are searched for and if found FGFR is inhibited and the PI3K/AKT/mTOR and MEK/RAS/RAF/ERK pathways are targeted for treatment of colorectal cancer with liver metastasis. In embodiments, FGFR mutations are searched for and if found the MEK/RAS/RAF/ERK pathway is targeted by an inhibitor. In embodiments, FGFR mutations are searched for and if found the MEK/RAS/RAF/ERK pathway is targeted by an activator. In embodiments, FGFR mutations are searched for and if found the PI3K/AKT/mTOR pathway is targeted by an inhibitor. In embodiments, FGFR mutations are searched for and if found the the PI3K/AKT/mTOR pathway is targeted by an activator. In embodiments, FGFR mutations are searched for and if found both the MEK/RAS/RAF/ERK and the PI3K/AKT/mTOR pathways are targeted by an inhibitor. In embodiments, FGFR mutations are searched for and if found both the MEK/RAS/RAF/ERK and the PI3K/AKT/mTOR pathways are targeted by an activator. In embodiments, FGFR mutations are searched for and if found the MEK/RAS/RAF/ERK pathway is targeted by an activator and the PI3K/AKT/mTOR pathway is targeted by an inhibitor. In embodiments, FGFR mutations are searched for and if found the MEK/RAS/RAF/ERK pathway is targeted by an inhibitor and the PI3K/AKT/mTOR pathway is targeted by an activator.

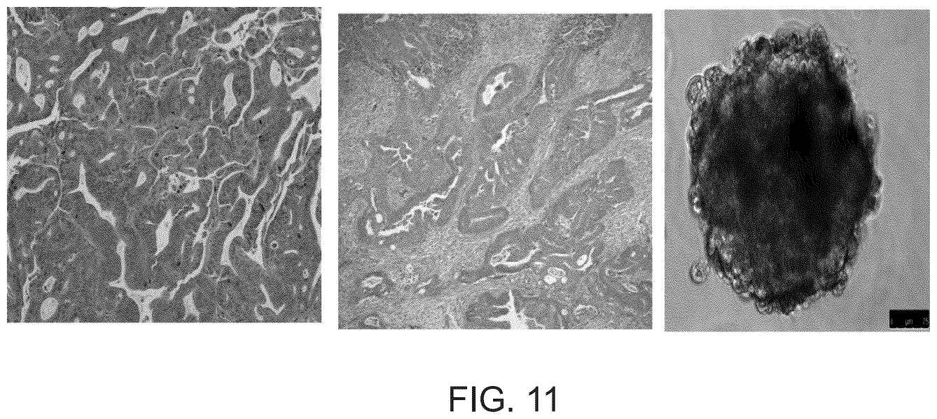

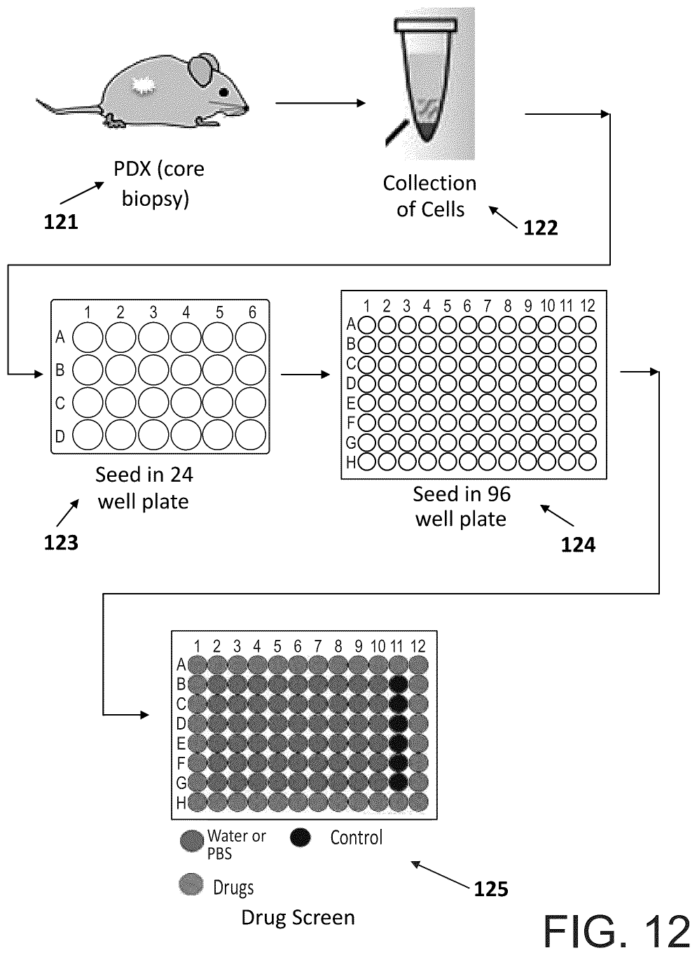

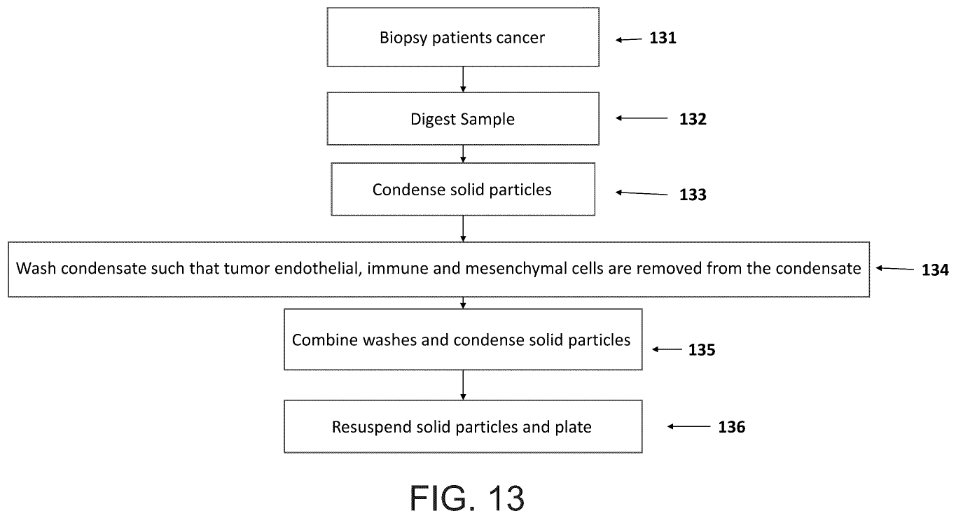

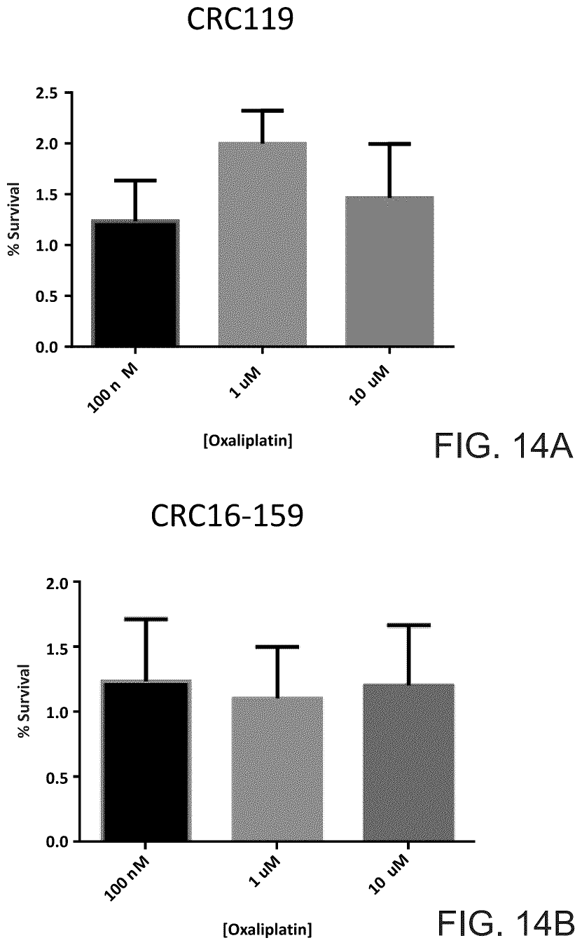

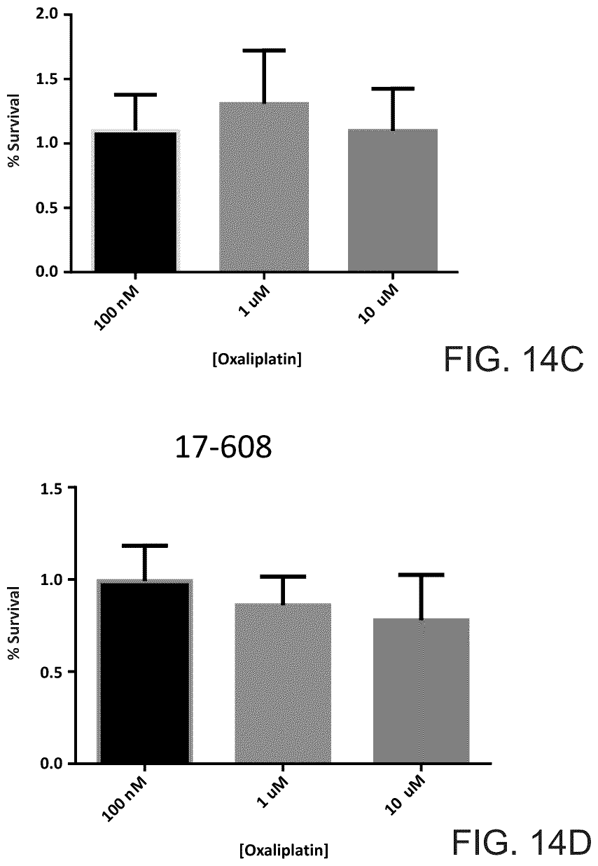

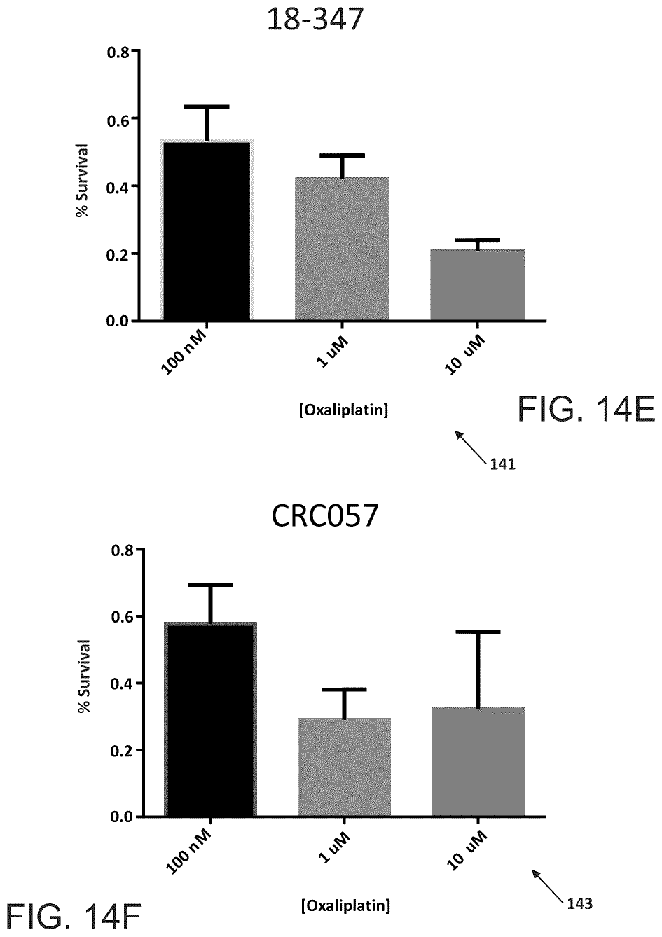

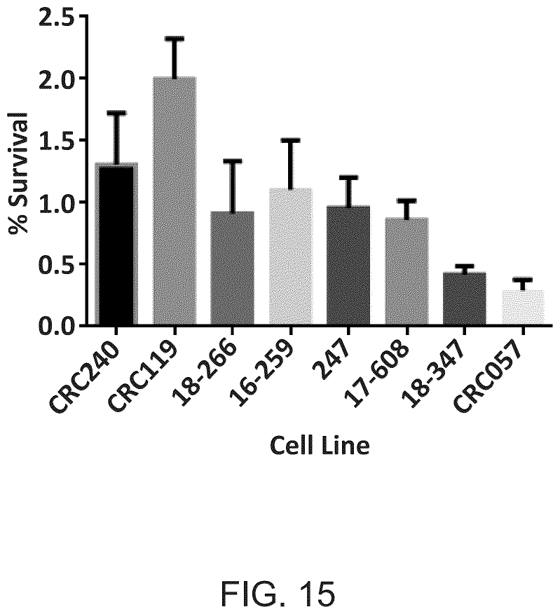

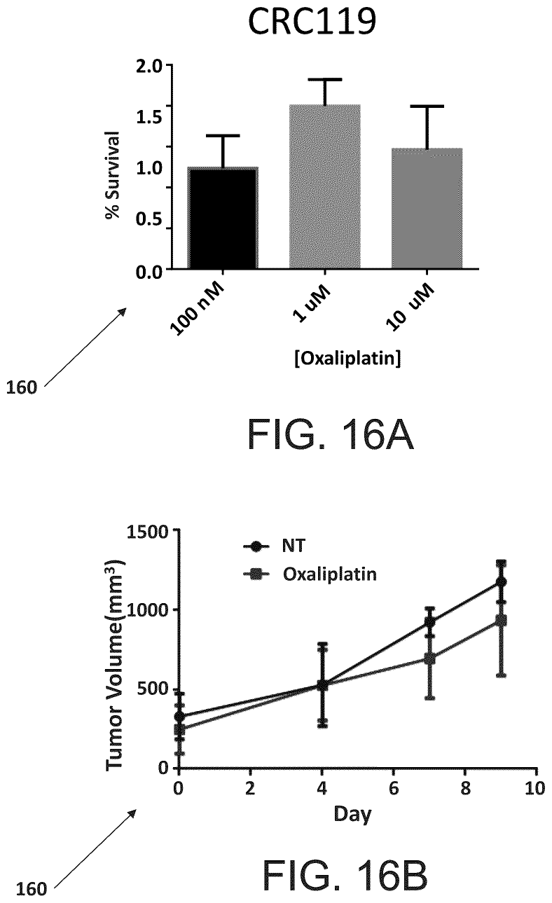

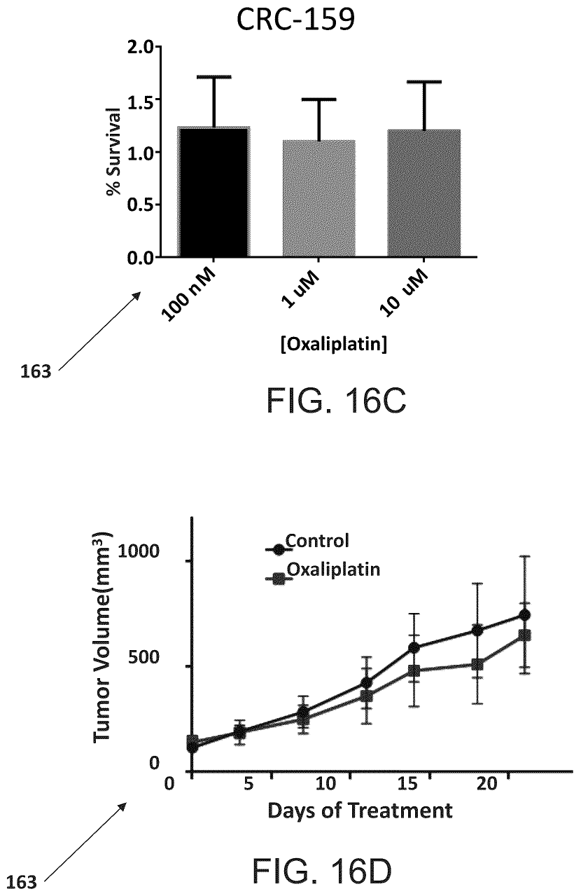

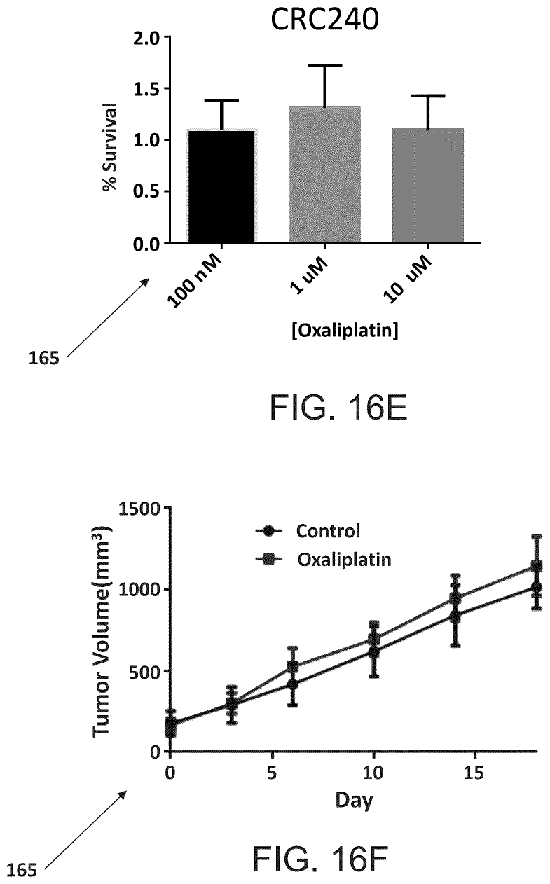

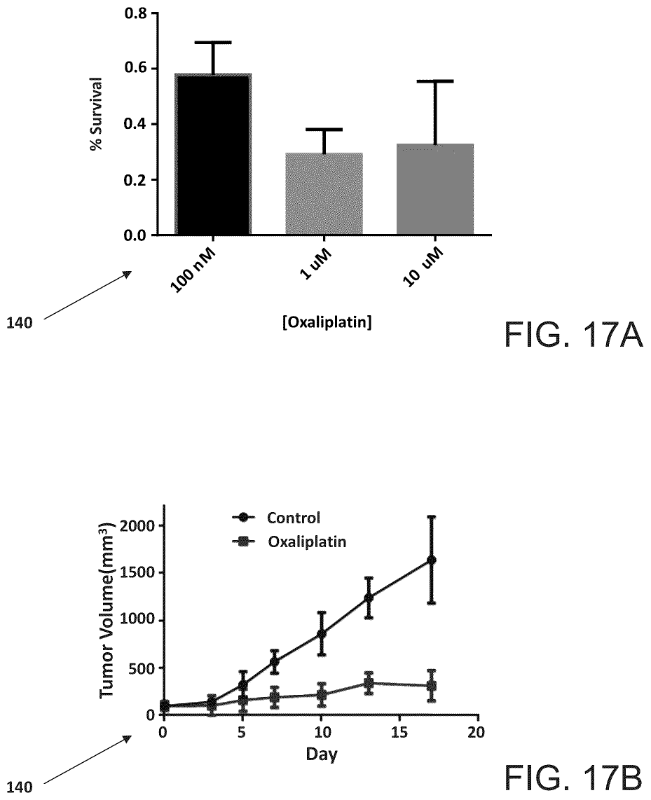

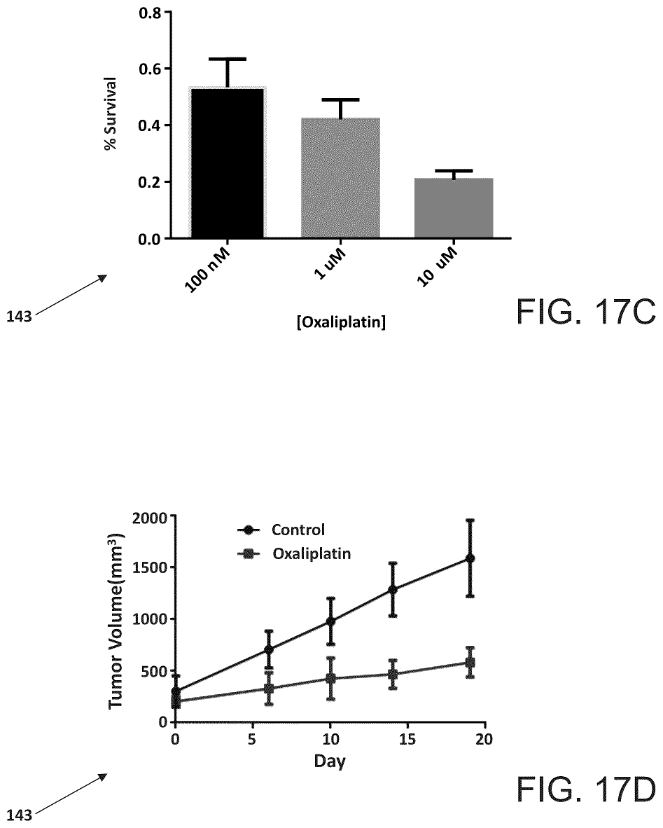

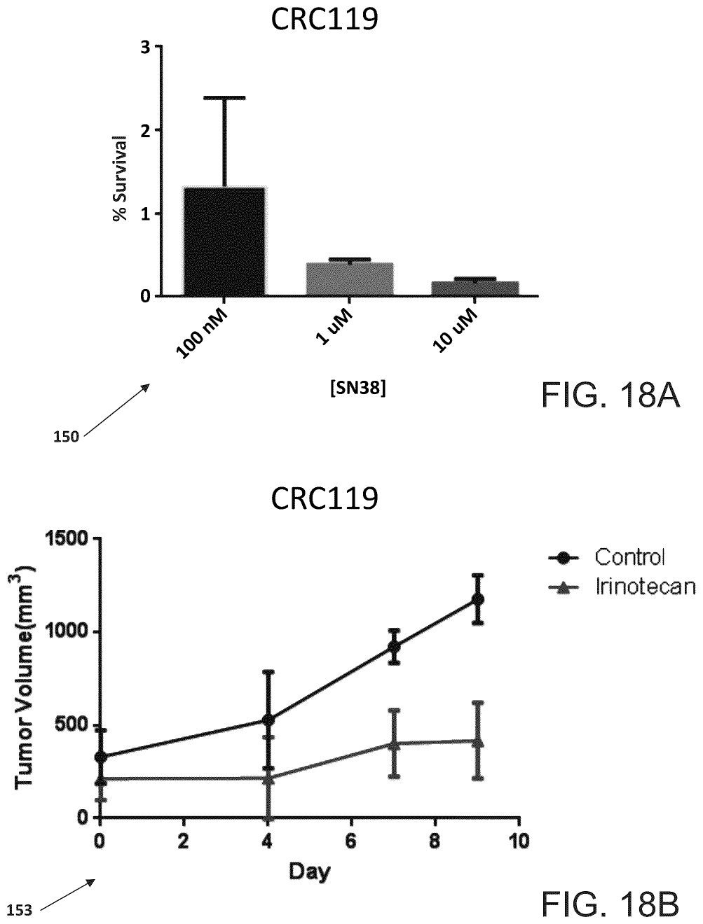

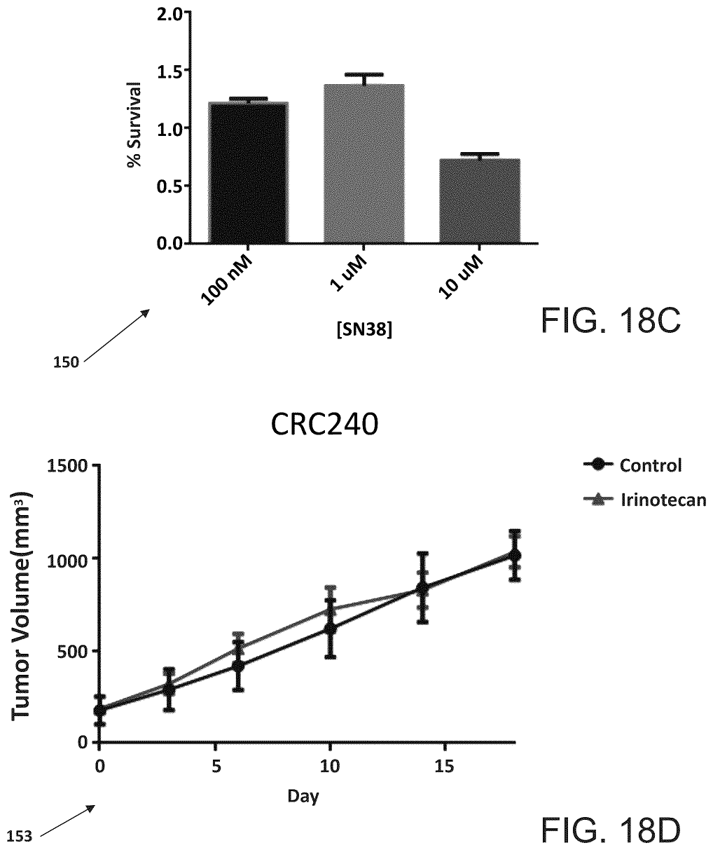

In embodiments, patients undergo biopsy of metastatic cancer, CT of the chest, abdomen and pelvis. In embodiments, drug sensitivity is tested within 7-10 days (or 2-3 days) of obtaining tissue. Histological features of an example patient tumor, matching organoids and PDX are shown in FIG. 11. In embodiments, FIG. 12, organoids are prepared from a PDX biopsy 121 by collecting and digesting cells 122, seeding the cells in a 24 well plate 123, after incubation seeding cells from the 24 well plate in a 96 well plate 124 and screening drugs 125. In embodiments, as shown in FIG. 13, a patients cancer is biopsied 131, the sample is digested 132, solid particles are condensed 133, the condensate is washed 134, washes are combined and solid particles condensed 135, and solid particles are resuspended and plated 136. Sensitivity to oxaliplatin at 100 nM, 1 μM, and 10 μM was tested on 8 organoids, CRC057, CRC119, CRC240, CRC16-159, CRC17-608, CRC18-347, CRC247 and CRC18-266, created using the above procedure. As shown in FIG. 14B, CRC18-347 141 and CRC057 143 were the only organoids found to have greater 50% killing at 1 μM. The sensitivity of all 8 cell lines studied to oxaliplatin is shown in FIG. 15. The organoid data was validated by testing the sensitivity of the same cell lines to oxaliplatin (FIGS. 16 and 17) and irinotecan (FIG. 18) in PDX models. As shown in FIG. 16, CRC119 160, CRC16-159 163, and CRC240 165 were resistant to oxaliplatin in both organoid A and PDX B tests. As shown in FIG. 17, CRC057 and 18-347 were found to be sensitive to oxaliplatin in both the organoids 170 and PDXs 173. SN38 (7-ethyl-10-hydroxycamptothecin) was tested, rather than irinotecan, in organoids since irinotecan undergoes deesterification to SN-38 in vivo but not in vitro. As shown in FIG. 18, CRC119 A and CRC240 B is sensitive to irinotecan in both organoids 180 and PDXS 183.

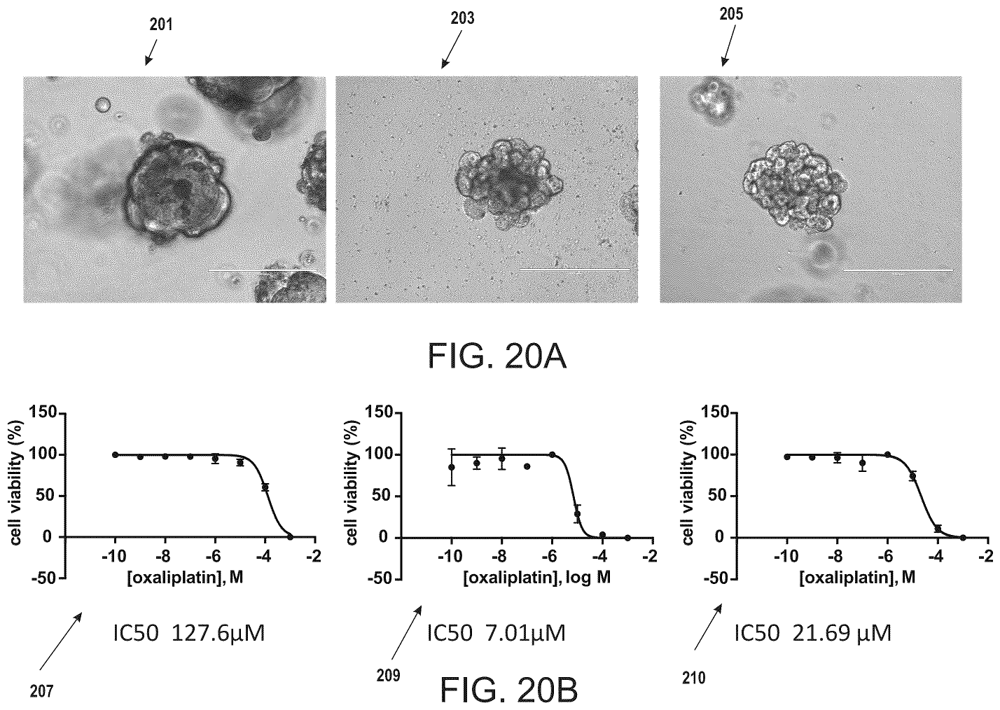

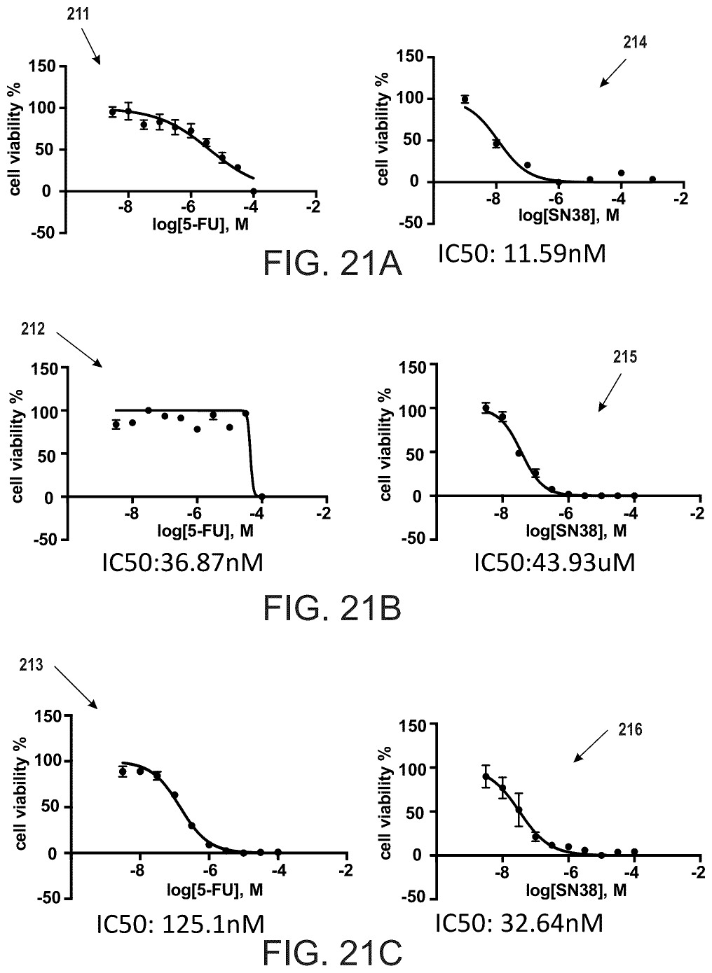

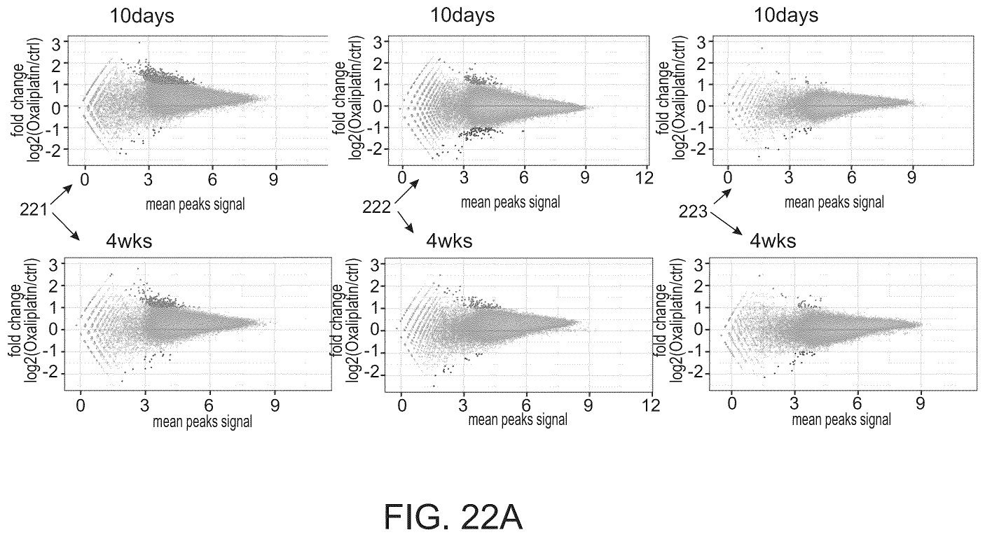

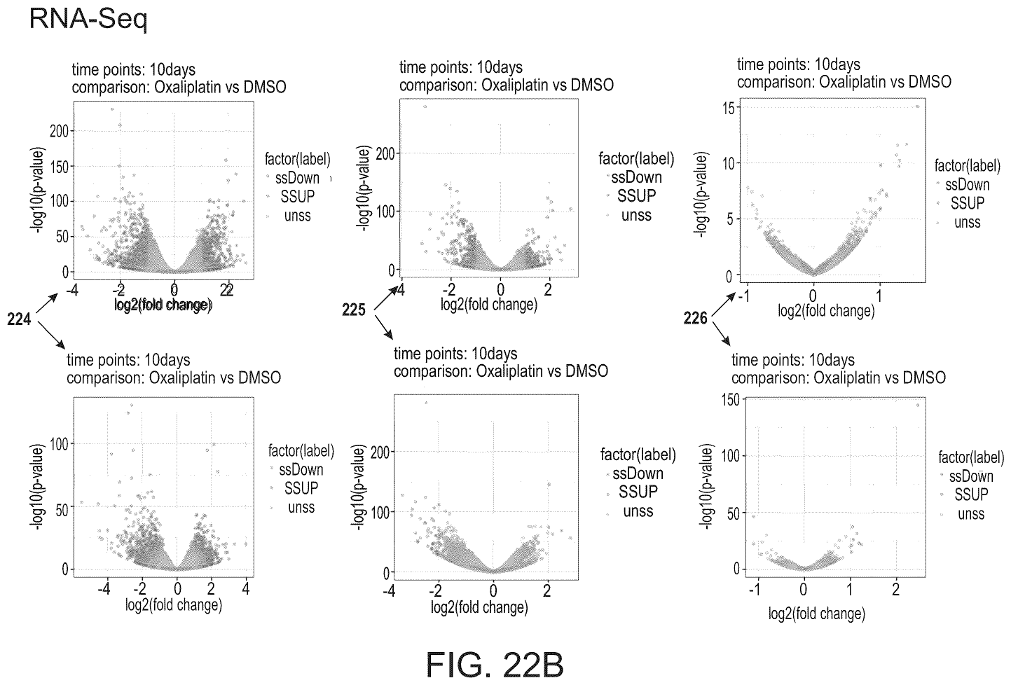

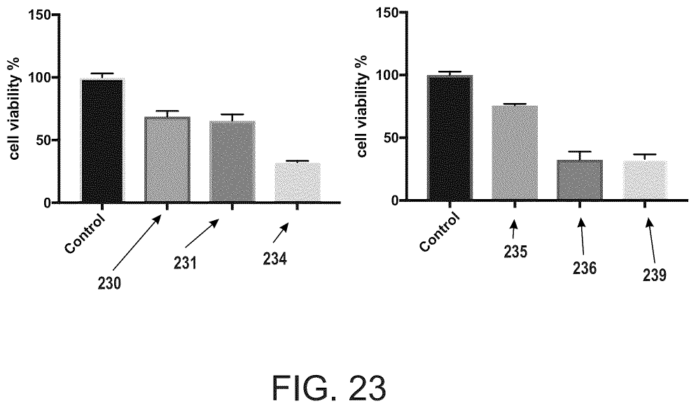

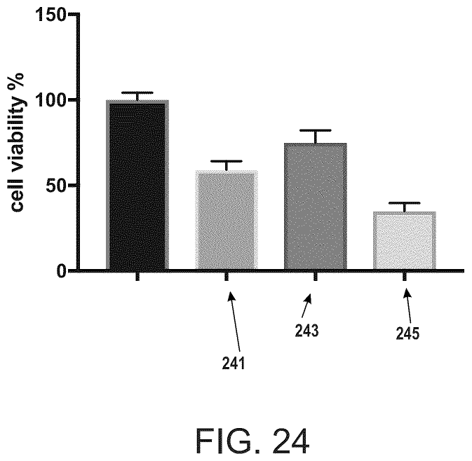

In an embodiment three organoids A 201 B 203 and C 205 were created as shown in FIG. 20A. The oxaliplatin IC50 for A B and C was 127.6 μM 207, 7.01 μM 209, and 21.69 μM 210 respectively as shown in FIG. 20B. The IC50s for fluorouracil (5FU) were 3.96 μM 211, 36.97 nM 212 and 125.1 nM 213 for A B and C respectively. The IC50s for SN38 were 11.59 nM, 214 43.93 μM 215 and 32.64 nM 216 for A B and C respectively as shown in FIG. 21. ATAC-Seq tests were run to determine which pathways were up and down regulated in the presence of various drugs. This data is shown in FIG. 22A for 10 days and 4 wks of treatment for A 221, B 222, and C 223 respectively. The ATAC Seq data was confirmed by RNA-Seq data as shown in FIG. 22B for A 224, B 225 and C 226. As can be seen from the IC50 data in FIG. 20 Organoid A was resistant to oxaliplatin. The ATAC Seq and RNA Seq data unexpectedly showed that the FGFR1 and oxytocin receptors were highly upregulated in the oxaliplatin resistance organoid. The effectiveness of oxaliplatin 230, an FGFR1 inhibitor 231, along with oxaliplatin and an FGFR1 inhibitor 234 as cell killers was tested in the organoid as shown in FIG. 23. The organoid data was confirmed in PDX models as shown in 235, 236, and 239, respectively. Paring oxaliplatin with an FGFR1 inhibitor achieved a synergistic effect. The cell killing potential of oxaliplatin 241, an oxytocin antagonist 243, along with oxaliplatin and an oxytocin antagonist 245 was tested as shown in FIG. 24. A similar synergistic effect was seen here. A PDX model is expected to give the same results due to the effectiveness of organoids at mimicking natural tumor conditions as described above.

In embodiments oxaliplatin resistant cancer is treated with an FGFR1inhibitor. In embodiments oxaliplatin resistant cancer is treated with an oxytocin antagonist. In embodiments oxaliplatin resistant cancer is treated with an FGFR1 inhibitor and an oxytocin antagonist. In embodiments oxaliplatin resistant cancer is treated with oxaliplatin and an FGFR1 inhibitor. In embodiments oxaliplatin resistant cancer is treated with oxaliplatin and an oxytocin antagonist. In embodiments oxalipatin resistant cancer is treated with oxaliplatin, an FGFR1 inhibitor, and an oxytocin antagonist. In embodiments oxaliplatin resistant colon cancer is treated with an oxytocin antagonist. In embodiments oxaliplatin resistant colon cancer is treated with an FGFR1 inhibitor. In embodiments oxaliplatin resistant colon cancer is treated with an FGFR1 inhibitor and an oxytocin antagonist. In embodiments oxaliplatin resistant colon cancer is treated with oxaliplatin and an FGFR1 inhibitor. In embodiments oxaliplatin resistant colon cancer is treated with oxaliplatin and an oxytocin antagonist. In embodiments oxaliplatin resistant colon cancer is treated with oxaliplatin, an FGFR1 inhibitor, and an oxytocin antagonist. In embodiments oxalipatin resistant colon cancer with liver metastasis is treated with an oxytocin antagonist. In embodiments oxalipatin resistant colon cancer with liver metastasis is treated with an FGFR1 inhibitor. In embodiments oxalipatin resistant colon cancer with liver metastasis is treated with an FGFR1 inhibitor and an oxytocin antagonist. In embodiments oxaliplatin resistant colon cancer with liver metastasis is treated with oxaliplatin and an FGFR1 inhibitor. In embodiments oxaliplatin resistant colon cancer with liver metastasis is treated with oxaliplatin and an oxytocin antagonist. In embodiments oxalipatin resistant colon cancer with liver metastasis is treated with oxaliplatin, an FGFR1 inhibitor, and an oxytocin antagonist.

The present subject matter may be a system, a method, and/or a computer program product. The computer program product may include a computer readable storage medium (or media) having computer readable program instructions thereon for causing a processor to carry out aspects of the present subject matter.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

Aspects of the present subject matter are described herein with reference to flowchart illustrations and/or block diagrams of methods, apparatus (systems), and computer program products according to embodiments of the subject matter. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of exemplary implementations of systems, methods, and computer program products according to various embodiments of the present subject matter. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the embodiments have been described in connection with the various embodiments of the various figures, it is to be understood that other similar embodiments may be used, or modifications and additions may be made to the described embodiment for performing the same function without deviating therefrom. Therefore, the disclosed embodiments should not be limited to any single embodiment, but rather should be construed in breadth and scope in accordance with the appended claims.

### REFERENCES

All of the below are incorporated by reference in their entirety.
1. Siegel RL, Miller KD, Jemal A. Cancer Statistics, 2017. CA: a cancer journal for clinicians 2017; 67:7-30.
2. Andre T, Bensmaine MA, Louvet C, et al. Multicenter phase II study of bimonthly high-dose leucovorin, fluorouracil infusion, and oxaliplatin for metastatic colorectal cancer resistant to the same leucovorin and fluorouracil regimen. Journal of clinical oncology: official journal of the American Society of Clinical Oncology 1999; 17:3560-8.
3. August DA, Sugarbaker PH, Ottow RT, Gianola FJ, Schneider PD. Hepatic resection of colorectal metastases. Influence of clinical factors and adjuvant intraperitoneal 5-fluorouracil via Tenckhoff catheter on survival. Annals of surgery 1985; 201:210-8.
4. Stangl R, Altendorf-Hofmann A, Charnley RM, Scheele J. Factors influencing the natural history of colorectal liver metastases. Lancet 1994; 343:1405-10.
5. Hurwitz H, Fehrenbacher L, Novotny W, et al. Bevacizumab plus irinotecan, fluorouracil, and leucovorin for metastatic colorectal cancer. The New England journal of medicine 2004; 350:2335-42.
6. Tournigand C, Andre T, Achille E, et al. FOLFIRI followed by FOLFOX6 or the reverse sequence in advanced colorectal cancer: a randomized GERCOR study. Journal of clinical oncology: official journal of the American Society of Clinical Oncology 2004; 22:229-37.
7. Fernandez FG, Drebin JA, Linehan DC, Dehdashti F, Siegel BA, Strasberg SM. Five-year survival after resection of hepatic metastases from colorectal cancer in patients screened by positron emission tomography with F-18 fluorodeoxyglucose (FDG-PET). Annals of surgery 2004; 240:438-47; discussion 47-50.
8. Fong Y, Fortner J, Sun RL, Brennan MF, Blumgart LH. Clinical score for predicting recurrence after hepatic resection for metastatic colorectal cancer: analysis of 1001 consecutive cases. Annals of surgery 1999; 230:309-18; discussion 18-21.
9. Fong Y, Cohen AM, Fortner JG, et al. Liver resection for colorectal metastases. Journal of clinical oncology: official journal of the American Society of Clinical Oncology 1997; 15:938-46.
10. Fortner JG. Recurrence of colorectal cancer after hepatic resection. American journal of surgery 1988; 155:378-82.
11. Hughes K, Scheele J, Sugarbaker PH. Surgery for colorectal cancer metastatic to the liver. Optimizing the results of treatment. The Surgical clinics of North America 1989; 69:339-59.
12. Barretina J, Caponigro G, Stransky N, et al. The Cancer Cell Line Encyclopedia enables predictive modelling of anticancer drug sensitivity. Nature 2012; 483:603-7.
13. van de Wetering M, Francies HE, Francis JM, et al. Prospective derivation of a living organoid biobank of colorectal cancer patients. Cell 2015; 161:933-45.
14. Gao H, Korn JM, Ferretti S, et al. High-throughput screening using patient-derived tumor xenografts to predict clinical trial drug response. Nature medicine 2015; 21:1318-25.
15. Lu M, Zessin AS, Glover W, Hsu DS. Activation of the mTOR Pathway by Oxaliplatin in the Treatment of Colorectal Cancer Liver Metastasis. PloS one 2017; 12:e0169439.
16. Pauli C, Hopkins BD, Prandi D, et al. Personalized In Vitro and In Vivo Cancer Models to Guide Precision Medicine. Cancer discovery 2017; 7:462-77.
17. Vlachogiannis G, Hedayat S, Vatsiou A, et al. Patient-derived organoids model treatment response of metastatic gastrointestinal cancers. Science 2018; 359:920-6.
18. Uronis JM, Osada T, McCall S, et al. Histological and molecular evaluation of patient-derived colorectal cancer explants. PloS one 2012; 7:e38422.
19. Kim MK, Osada T, Barry WT, et al. Characterization of an oxaliplatin sensitivity predictor in a preclinical murine model of colorectal cancer. Molecular cancer therapeutics 2012; 11:1500-9.
20. Suggitt M, Bibby MC. 50 years of preclinical anticancer drug screening: empirical to target-driven approaches. Clin Cancer Res 2005; 11:971-81.
21. Fichtner I, Slisow W, Gill J, et al. Anticancer drug response and expression of molecular markers in early-passage xenotransplanted colon carcinomas. European journal of cancer 2004; 40:298-307.
22. Dangles-Marie V, Pocard M, Richon S, et al. Establishment of human colon cancer cell lines from fresh tumors versus xenografts: comparison of success rate and cell line features. Cancer research 2007; 67:398-407.
23. Guenot D, Guerin E, Aguillon-Romain S, et al. Primary tumour genetic alterations and intra-tumoral heterogeneity are maintained in xenografts of human colon cancers showing chromosome instability. The Journal of pathology 2006; 208:643-52.
24. Bertotti A, Migliardi G, Galimi F, et al. A molecularly annotated platform of patient-derived xenografts (“xenopatients”) identifies HER2 as an effective therapeutic target in cetuximab-resistant colorectal cancer. Cancer discovery 2011; 1:508-23.
25. Tentler JJ, Nallapareddy S, Tan AC, et al. Identification of predictive markers of response to the MEK1/2 inhibitor selumetinib (AZD6244) in K-ras-mutated colorectal cancer. Molecular cancer therapeutics 2010; 9:3351-62.
26. Uronis J, Osada, T, McCall,S., Yang, X., Mantyh, C., Morse,M., Lyerly, K., Clary, B., and Hsu, D.S. Histological and Molecular Evaluation of Patient-Derived Colorectal Cancer Explants PloS one 2012; accepted for publication (May 10, 2012).
27 Tentler JJ, Tan AC, Weekes CD, et al. Patient-derived tumour xenografts as models for oncology drug development. Nature reviews Clinical oncology 2012; 9:338-50.
28. Pauli C, Hopkins BD, Prandi D, et al. Personalized <em>In Vitro</em> and <cm>In Vivo</em> Cancer Models to Guide Precision Medicine. Cancer Discovery 2017; 7:462-77.
29. Douillard JY, Siena S, Cassidy J, Tabernero J, Burkes R, Barugel M, et al. Randomized phase III trial of panitumumab with infusional fluorouracil, leucovorin, and oxaliplatin (FOLFOX4) versus FOLFOX4 alone as a first-line treatment in patients with previously untreated metastatic colorectal cancer: the PRIME study. J Clin Oncol 2010; 28:4697-705.
30. Saltz LB, Clarke S, Diaz-Rubio E, Scheithauer W, Figer A, Wong R, et al. Bevacizumab in combination with ozaliplatin-based chemotherapy as a first-line therapy in metastastic colorectal cancer: a randomized phase III study. J Clin Oncol 2008; 26:2013-9.
31. Arrowsmith J. Trial watch: phase III and submission failures: 2007-2010. Nature reviews Drug discovery 2011; 10:87.
32. Cingolani P, Platts A, Wang LL, Coon M, Nguyen T, Wang L, et al. A program for annotating and predicting the effects of single nucleotide polymorphisms, SnpEff: SNPs in the genome of Drosophila melanogaster strain w(1118); iso-2; iso-3. Fly 2012; 6:80-92.
33. O'Hare T, Shakespeare WC, Zhu X, Eide CA, Rivera VM, Wang F, et al. AP24534, a Pan-BCR-ABL Inhibitor for Chronic Myeloid Leukemia, Potently Inhibits the T315I Mutant and Overcomes Mutation-Based Resistance. Cancer cell 2009; 16:401-12.
34. Shah NP, Lee FY, Luo R, Jiang Y, Donker M, Akin C. Dasatinib (BMS-354825) inhibits KIT<sup>D816V</sup>, an imatinib-resistant activating mutation that triggers neoplastic growth in most patients with systemic mastocytosis. Blood 2006; 108-286-91.
35. Gozgit JM, Wong MJ, Moran L, Wardwell S, Mohemmad QK, Narasimhan NI, et al. Ponatinib (AP24534), a Multitargeted Pan-FGFR Inhibitor with Activity in Multiple FGFR-Amplified or Mutated Cancer Models. Molecular Cancer Therapeutics 2012; 11:690-9.
36. Sun L, Liang C, Shirazian S, Zhou Y, Miller T, Cui J, et al. Discovery of 5-[5-Fluoro-2-oxo-1,2-dihydroindol-(3Z)-ylidenemethyl]-2,4-dimethyl-1H-pyrrole-3-carboxylic Acid (2-Diethylaminoethyl)amide, a Novel Tyrosine Kinase Inhibitor Targeting Vascular Endothelial and Platelet-Derived Growth Factor Receptor Tyrosine Kinase. Journal of Medicinal Chemistry 2003; 46:1116-9.
37. Hu-Lowe DD, Zou HY, Grazzini ML, Hallin ME, Wickman GR, Amundson K, et al. Nonclinical Antiangiogenesis and Antitumor Activities of Axitnib (AG-013736), an Oral, Potent, and Selective Inhibitor of Vascular Endothelial Growth Factor Receptor Tyrosine Kinases 1, 2, 3. Clinical Cancer Research 2008; 14:7272-83.
38. O'Hare T, Walters DK, Stoffregen EP, Jia T, Manley PW, Mestan J, et al. <em>In vitro</em> Activity of Bcr-Ab1 Inhibitors AMN107 and BMS-354825 against Clinically Relevant Imatinib-Resistant Ab1 Kinase Domain Mutants. Cancer Research 2005; 65:4500-5.
39. Touat M, LLeana E, Postel-Vinay S, Andre F, Soria J-C. Targeting FGFR Signaling in Cancer. Clinical Cancer Research 2015; 21:2684-94.
40. Chae YK, Ranganath K, Hammerman PS, Vaklavas C, Mohindra N, Kalyan A, et al. Inhibition of the fibroblast growth factor receptor (FGFR) pathway: the current landscape and barriers to clinical application. Oncotarget 2017; 8:16052-74.
41. Guagnano V, Kauffmann A, Wohrle S, Stamm C, Ito M, Barys L, et al. FGFR Genetic Alterations Predict for Sensitivity to NVP-BGJ398, a Selective Pan-FGFR Inhibitor Cancer Discovery 2012; 2:1118-33.
42. Weiss J, Sos ML, Seidel D, Peifer M, Zander T, Heuckmann JM, et al. Frequent and focal FGFR1 amplification associates with therapeutically tractable FGFR1 dependency in squamous cell lung cancer. Science translational medicine 2010; 2:62ra93.
43. Courjal F, Cuny M, Simony-Lafontaine J, Louason G, Speiser P, Zeillinger R, et al. Mapping of DNA amplifications at 15 chromosomal localizations in 1875 breast tumors: definition of phenotypic groups. Cancer research 1997; 57:4360-7.
44. Turner N, Pearson A, Sharpe R, Lambros M, Geyer F, Lopez-Garcia MA, et al. FGFR 1 amplification drives endocrine therapy resistance and is a therapeutic target in breast cancer. Cancer research 2010; 70:2085-94.
45. Babina IS, Turner NC. Advances and challenges in targeting FGFR signaling in cancer. Nature reviews Cancer 2017; 17:318-32.
46. Singh D, Chan JM, Zoppoli P, Niola F, Sullivan R, Castano A, et al. Transforming fusions of FGFR and TACC genes in human glioblastoma. Science 2012; 337:1231-5.
47. Wu YM, Su F, Kalyana-Sundaram S, Khazanov N, Ateeq B, Cao X, et al. Identification of targetable FGFR gene fusions in diverse cancers. Cancer discovery 2013; 3636-47.
48. Karkera JD, Cardona GM, Bell K, Gaffney D, Portale JC, Santiago-Walker A, et al. Oncogenic Characterization and Pharmacologic Sensitivity of Activating Fibroblast Growth Factor Receptor (FGFR) Genetic Alterations to the Selective FGFR Inhibitor Erdafitinib. Molecular cancer therapeutics 2017; 16:1717-26.
49. Sonvilla G, Allerstorfer S, Heinzle C, Stattner S, Karner J, Klimpfinger M, et al. Fibroblast growth factor receptor 3-IIIc mediates colorectal cancer growth and migration. British journal of cancer 2010; 102:1145-56.
50. Kwak Y, Nam SK, Seo AN, Kim DW, Kang SB, Kim WH, et al. Fibroblast Growth Factor Receptor 1 Gene Copy Number and mRNA Expression in Primary Colorectal Cancer and Its Clinicopathologic Correlation Pathobiology: journal of immunopathology, molecular and cellular biology 2015; 82:76-83.
51. Bange J, Prechtl D, Cheburkin Y, Specht K, Harbeck N, Schmitt M, et al. Cancer progression and tumor cell motility are associated with the FGFR4 Arg(388) allele. Cancer research 2002; 62:840-7.
52. Spinola M, Leoni VP, Tanuma J, Pettinicchio A, Frattini M, Signoroni S, et al. FGFR4 Gly388Arg polymorphism and prognosis of breast and colorectal cancer. Oncology reports 2005; 14:415-9.

1.-24. (canceled)

25. A method of treatment for a patient comprising:
generating a patient specific tumor model comprising an organoid;
wherein the organoid is prepared by obtaining a biopsy of tissue from the patient's tumor;
digesting cells from the biopsied tissue;
seeding the cells in a Matrigel culture medium to generate the organoid,
wherein the organoid comprises tumor immune, endothelial and mesenchymal cells;
testing one or more drugs on the patient specific tumor model wherein the model is generated and the one or more drugs are tested on the model within 3 days of acquiring the patient biopsy; and
treating the patient based on the results of the patient specific tumor model tests.

26. The method of claim 25, wherein the patient's tumor is a colorectal cancer tumor.

27. The method of claim 25, further comprising:
entering patient specific information into a computational model; and
treating a patient based on the results of the patient specific tumor model tests and the computational model.

28. The method of claim 25, wherein the patient specific information comprises at least one of biopsy immunohistochemistry (IHC), biopsy sequencing data, biomarkers, diagnostic information, genetic mutations present in the tumor, medical images, histology images, immunohistochemistry images, patient disease progression throughout treatment, and results of patient specific tumor model tests.

29. The method of claim 25, wherein the tested drug comprises oxaliplatin.

30. A clinical trial system comprising:
generating a patient specific tumor model comprising an organoid;
wherein the organoid is prepared by obtaining a biopsy of tissue from a patient's tumor;
digesting cells from the biopsied tissue;
seeding the cells in a Matrigel culture medium to generate the organoid,
wherein the organoid comprises tumor immune, endothelial and mesenchymal cells;
testing one or more drugs on the patient specific tumor model wherein the model is generated and the one or more drugs are tested on the model within 3 days of acquiring the patient biopsy; and
treating a patient based on the results of the patient specific tumor model tests.

31. The clinical trial system of claim 30, wherein the patient's tumor is a colorectal cancer tumor.

32. The clinical trial system of claim 30, further comprising:
entering patient specific information into a computational model; and
treating a patient based on the results of the patient specific tumor model tests and the computational model.

33. The clinical trial system of claim 30, wherein the patient specific information comprises at least one of biopsy immunohistochemistry (IHC), biopsy sequencing data, biomarkers, diagnostic information, genetic mutations present in the tumor, medical images, histology images, immunohistochemistry images, patient disease progression throughout treatment, and results of patient specific tumor model tests.

34. The clinical trial system of claim 30, wherein the tested drug comprises oxaliplatin.

35. The clinical trial system of claim 30, wherein the model is generated and the one or more drugs are tested on the model within 2-3 days of acquiring the patient biopsy.

36. The clinical trial system of claim 30, wherein the organoids are implemented in a 2-D monolayer culture.

37. The clinical trial system of claim 30, wherein isolated patient blood or T cells are added to the organoid.

38. The clinical trial system of claim 30, wherein a CRISPR screen with pooled guide RNAs is conducted.

39. A method of creating a patient-derived tumor organoid, the method comprising:
obtaining a biopsy of tissue from a patient's tumor;
digesting cells from the biopsied tissue; and
seeding the cells in a Matrigel culture medium wherein tumor immune, endothelial and mesenchymal cells are included in the organoid and wherein the organoid is ready for drug sensitivity testing on the organoid within 3 days after obtaining the biopsy.

40. The method of claim 39, wherein the patient's tumor is a colorectal cancer tumor.

41. An organoid comprising tumor immune, tumor endothelial and tumor mesenchymal cells from a patient tumor biopsy, a Matrigel culture medium, and wherein the organoid is ready for drug sensitivity testing on the organoid within 3 days after obtaining the biopsy.

42. The organoid of claim 41, wherein the patient's tumor is a colorectal cancer tumor.

43. The method of claim 41, wherein less than 2 mm³ of tissue is digested.

44. (canceled)


SEQUENCE LISTING (6 sequences)

SEQ ID NO: 1 - DNA, 18 bp, Homo sapiens
  source 1..18: mol_type = other DNA
atgctggccgctacctct

SEQ ID NO: 2 - DNA, 19 bp, Homo sapiens
  source 1..19: mol_type = other DNA
gacttgccggaagagcctg

SEQ ID NO: 3 - DNA, 19 bp, Homo sapiens
  source 1..19: mol_type = other DNA
atgctggccgctacctctg

SEQ ID NO: 4 - DNA, 20 bp, Homo sapiens
  source 1..20: mol_type = other DNA
gacttgccggaagagcctga

SEQ ID NO: 5 - DNA, 17 bp, Homo sapiens
  source 1..17: mol_type = other DNA
ggccgctacctctgcct

SEQ ID NO: 6 - DNA, 20 bp, Homo sapiens
  source 1..20: mol_type = other DNA
gcttgacttgccggaagagc